(19) United States
(12) Patent Application Publication
(10) Pub. No.: US 2022/0287995 A1
(43) Pub. Date: Sep. 15, 2022

(54) COMPOSITIONS AND METHODS FOR INHIBITING BACTERIAL VIRULENCE AND FLIM-BASED DEVICE AND METHOD FOR ANTIBIOTIC SUSCEPTIBILITY TESTING

(71) Applicant: THE REGENTS OF THE UNIVERSITY OF CALIFORNIA, OAKLAND, CA (US)

(72) Inventors: Albert SIRYAPORN, IRVINE, CA (US); Michelle DIGMAN, IRVINE, CA (US); Jenu VARGHESE CHACKO, IRVINE, CA (US); Kumar PERINBAM, IRVINE, CA (US); Anerudh KANNAN, IRVINE, CA (US); Allon HOCHBAUM, IRVINE, CA (US); Katrine WHITESON, IRVINE, CA (US)

(73) Assignee: THE REGENTS OF THE UNIVERSITY OF CALIFORNIA, OAKLAND, CA (US)

(21) Appl. No.: 17/597,922

(22) PCT Filed: Jul. 30, 2020
(86) PCT No.: PCT/US20/44301

§ 371 Date: Jan. 28, 2022

Related U.S. Application Data
(60) Provisional application No. 62/880,375, filed on Jul. 30, 2019.

(51) Int. Cl. A61K 31/194; A61K 31/19; C12Q 1/02

(52) U.S. Cl. CPC A61K 31/194; A61K 31/19; C12Q 1/025

(57) ABSTRACT

Compositions and methods for inhibiting bacterial virulence, as well as methods and materials for use in rapid assessment of antibiotic susceptibility are described. A method for inhibiting bacterial virulence comprises exposing a site containing or suspected of containing virulent bacteria to a carbon source, wherein the carbon source produces a low g value. Examples of such carbon sources include pyruvate, citrate, oxaloacetate, malate, and fumarate. The carbon source can be applied to a surface or administered to a subject. A device for testing antibiotic susceptibility of bacteria comprises a fluorescence lifetime imaging microscopy (FLIM) apparatus that emits an excitation pulse of light directed at a receiving surface; a detector that collects time-correlated fluorescence emitted from individual bacteria immobilized on the receiving surface; an analyzer that generates a FLIM-phasor profile; and an analyzer that correlates the FLIM-phasor profile to the status of the antibiotic susceptibility of the bacteria.

This application claims benefit of U.S. provisional patent application No. 62/880,375, filed Jul. 30, 2019, the entire contents of which are incorporated by reference into this application.

### ACKNOWLEDGEMENT OF GOVERNMENT SUPPORT

This invention was made with Government support under Grant Nos. AI112816, AI139968, and GM076516, awarded by the National Institutes of Health (NIH). The Government has certain rights in the invention.

### BACKGROUND OF THE INVENTION

Pseudomonas aeruginosa is an opportunistic pathogen that is responsible for a range of illnesses including lung infection in cystic fibrosis patients, hospital-acquired infections, sepsis, and disease in immunocompromised patients^{1,2}. This bacterium infects a broad range of hosts including humans, animals, plants, insects, amoebae, and other bacteria using a multitude of virulence factors including type III secretion, cyanide, pyocyanin, and proteases³⁻⁶.

There remains a need for methods of inhibiting bacterial virulence, and particularly for combatting virulence of P. aeruginosa. There also remains a need for methods and devices capable of rapid assessment of antibiotic susceptibility.

### SUMMARY OF THE INVENTION

The methods described herein address these needs and more by providing compositions and methods for inhibiting bacterial virulence, as well as methods and materials for use in rapid assessment of antibiotic susceptibility. In one embodiment, the invention provides a method for inhibiting bacterial virulence comprising exposing a site containing or suspected of containing virulent bacteria to a carbon source, wherein the carbon source produces a low g value. Also provided is a method for inhibiting bacterial virulence in a subject in need thereof. In one embodiment, the method comprises administering a carbon source to the subject, wherein the carbon source produces a low g value. In some embodiments, the bacteria comprise Pseudomonas aeruginosa.

The carbon source is typically administered to the subject by topical application, injection into a wound site, or intravenous administration. In some embodiments, the subject is a hospital or surgical patient. In some embodiments, the subject is intubated, catheterized, or on a respirator. In some embodiments, the subject is immunocompromised.

Examples of a carbon source include, but are not limited to, pyruvate or citrate. Other examples of a carbon source include, but are not limited to, oxaloacetate, malate, and fumarate. The carbon source is selected to produce a low g value, such as one that is less than cells in the virulent state. One example of a low g value is 0.3.

Also described herein is a device for testing antibiotic susceptibility of bacteria. In some embodiments, the device comprises: (a) a receiving surface adapted to receive and immobilize bacteria in contact with a test antibiotic; (b) a fluorescence lifetime imaging microscopy (FLIM) apparatus that emits an excitation pulse of light directed at the receiving surface; (c) a detector that collects time-correlated fluorescence emitted from individual bacteria immobilized on the receiving surface; (d) an analyzer that correlates time-correlated fluorescence emitted from individual bacteria collected by the detector with the excitation pulse emitted by the FLIM apparatus to generate a FLIM-phasor profile; and (e) an analyzer that correlates the FLIM-phasor profile generated in step (d) to the status of the antibiotic susceptibility of the bacteria. In some embodiments, the detector collects fluorescence with nanosecond resolution. In some embodiments, the analyzer performs a computational analysis algorithm that correlates time-correlated fluorescence emitted from bacteria collected by the detector to the status of the antibiotic susceptibility of the bacteria.

Additionally provided is a method of testing antibiotic susceptibility of bacteria isolated from a patient sample. In some embodiments, the method comprises: (a) immobilizing bacteria isolated from a patient sample onto a receiving surface; (b) contacting the immobilized bacteria with a test antibiotic and measuring the FLIM signatures at an initial time point and at regular intervals for 30 minutes to 1 hour. No addition of fluorophore is required. In some embodiments, the method further comprises: (c) directing a series of nanosecond excitation pulses of light at the immobilized bacteria; (d) collecting time-correlated fluorescence emitted from individual bacteria immobilized on the receiving surface; (e) generating FLIM-phasor profiles by taking the sine and cosine transform of the fluorescence intensity decays, thereby generating s and g values; and (f) comparing FLIM-phasor profiles obtained before and after the contacting of step (b); wherein a change in the g value upon contact with a test antibiotic indicates bacterial susceptibility to the test antibiotic. Pulses are directed at single bacteria and at bacteria in clusters. The change in the s and g values as a function of exposure time to the antibiotic will be computed to determine how the bacteria respond metabolically to the antibiotics. In some embodiments, the change is a statistically significant change.

In some embodiments, the test antibiotic is selected from the group consisting of: amoxicillin (penicillin-type), cephalexin (cephalosporin), erythromycin (macrolide), ciprofloxacin (fluoroquinolone), trimethoprim (sulfonamide), tetracycline, and gentamicin (aminoglycoside). In some embodiments, steps (c)-(e) of the method are repeated at intervals of 10-20 minutes for 1-3 hours after contacting the bacteria with test antibiotic. In some embodiments, steps (c)-(e) of the method are repeated at intervals of 15 minutes for 2 hours after contacting the bacteria with test antibiotic. In some embodiments, the method is completed in less than one hour.

Also provided is a system for testing antibiotic susceptibility of bacteria, the system comprising a user device comprising a hardware processor that is programmed to generate and analyze FLIM-phasor profiles as recited above. Additionally provided is a non-transitory computer-readable medium containing computer executable instructions that, when executed by a processor, cause the processor to generate and analyze FLIM-phasor profiles as recited above.

### BRIEF DESCRIPTION OF THE DRAWINGS

FIG. 1. Metabolic profiling of live planktonic P. aeruginosa cells using fluorescence lifetime imaging microscopy. (A) Fluorescence intensities (AU) and fluorescence lifetimes (nanoseconds) of an unlabeled P. aeruginosa cell imaged using the NADH emission spectrum. The corresponding intensity or lifetime histograms are displayed below the images. Arrows indicate clusters with relatively long fluorescence lifetimes. (B) Fluorescence intensities and lifetimes of multiple P. aeruginosa cells. (C) Phasor plot in which the cosine and sine components of the fluorescence lifetime are transformed into g and s coordinates, respectively. Each dot represents the fluorescence lifetime averaged over an individual P. aeruginosa cell. The metabolic trajectory of eukaryotic cells is plotted for reference using lifetime values of 0.4 ns and 3.4 ns for free and protein-bound NADH, respectively. Planktonic P. aeruginosa from three independent experiments were cultured to early exponential phase in modified minimal medium containing 0.2% citrate as the carbon source. Scale bars indicate 1 μm.

FIG. 2. Carbon sources alter NAD(H) concentrations and fluorescence lifetimes in surface-attached P. aeruginosa. (A) Schematic indicating the production of NADH by central metabolism pathways in P. aeruginosa. Measurements of (B) NADH/NAD+ ratios, (C) total NADH and NAD+ production, and (D) the concentration of NADH or NAD+ measured using an enzyme-cycling assay using surface-attached P. aeruginosa cultured using single carbon sources at 0.2%. Bars indicate the average and error bars indicate the standard deviation of three independent experiments. (E) Fluorescence lifetime phasor plot (left) and corresponding g-values (right) of P. aeruginosa cultured using identical conditions as the enzyme-cycling assay. Points on the map represent the average fluorescence lifetime of approximately five cells. The centers and axis lengths of the ellipses correspond to the mean and standard deviation, respectively, of all cells pooled from three independent experiments. The dashed line indicates a metabolic trajectory that connects the average phasor positions for different carbon sources. The arrow indicates the direction towards the position of the free NADH reference, which does not appear on this plot. The bars and errors bars in the bar graph indicate the means and standard deviations of the means of three independent experiments, respectively. (F) Total NAD(H) concentrations and fluorescence lifetime g-values for growth in single carbon sources. The points and errors bars indicate the means and standard deviations of the means of three independent experiments, respectively. The best-fit line is indicated with R=−0.94. All measurements were made on surface-attached P. aeruginosa that were cultured in modified minimal medium and harvested at mid-exponential phase, with the exception of the □phz1/2 strain, which was cultured in modified MOPS medium and harvested at different phases. ns—not significant.

FIG. 3. Virulence activation coincides with a growth transition. (A) An image-based assay using amoeba host cells and calcein-AM fluorescence (green) indicates the virulence of wild-type or ΔlasR P. aeruginosa and is used to compute host killing indexes. Scale bars indicate 25 μm. (B) Host killing indexes for planktonic or surface-attached sub-populations of wild-type, ΔlasR, and ΔpilY1 P. aeruginosa following 6 hours of growth from dilution of a saturated culture. (C) The host killing indexes of planktonic or surface-attached wild-type P. aeruginosa following 0, 2, 4, and 6 hours of growth. (D) Growth profiles measured using optical density (OD₆₀₀) and (E) fluorescence lifetimes of planktonic P. aeruginosa. For (B) through (D), bars and data points represent the average of three independent experiments and error bars indicate standard deviation. P. aeruginosa were cultured in rich (PS:DB) medium.

FIG. 4. Virulence-activated and low-virulence populations have distinct fluorescence lifetimes and NAD(H) concentrations. (A) Fluorescence lifetime phasor maps of surface-attached (red) or planktonic (blue) populations of wild-type (left panel), ΔlasR (middle panel), or ΔpilY1 (right panel) P. aeruginosa during the growth transition phase. Each data point represents a single P. aeruginosa cell. Data are pooled from 3 independent experiments. A vertical line at a g-value of 0.4 is plotted for reference. (B) Total NADH and NAD+ concentrations and corresponding (C) NADH/NAD+ ratios for surface-attached and planktonic wild-type P. aeruginosa during the growth transition phase. Bars indicate the mean of 3 independent experiments and error bars represent standard deviation. P. aeruginosa were cultured in rich (PS:DB) medium, with the exception of the Δphz1/2 strain, which was cultured in modified MOPS medium. *p<0.05, **p<0.01, ***p<0.001.

FIG. 5. Perturbing central metabolism inhibits or induces earlier activation of virulence. (A) Fluorescence lifetime phasor plot (left) and g-values (right) of surface-attached P. aeruginosa cultured in rich (PS:DB) medium, supplemented with carbon sources at 0.2% concentration at 3 hours following dilution, and harvested after an additional hour of growth. Each data point represents a single P. aeruginosa cell. The centers and axis lengths of the ellipses correspond to the mean and standard deviation, respectively, of all cells pooled from three independent experiments. The bars and errors bars in the bar graph indicate the means and standard deviations of the means of three independent experiments, respectively. (B) Total NADH and NAD+ concentrations and (C) corresponding NADH/NAD+ ratios for surface-attached P. aeruginosa cultured under identical conditions as in (A). (D) Host killing indexes for surface-attached wild-type or ΔlasR P. aeruginosa assessed after 6 hours of growth. Wild-type cultures were supplemented after 3 hours of growth with individual carbon sources or phosphoric acid, at 0.2% concentration. Bars are the average of three independent experiments and error bars represent standard deviation. (E) Host killing indexes of wild-type or ΔlasR surface-attached P. aeruginosa at 4.5 to 6 hours of growth following treatment at 3 hours with 0.1% ethanol (control) or 10 μM antimycin A dissolved in 0.1% ethanol. (F) Host killing indexes of wild-type surface-attached P. aeruginosa following treatment at 3 hours with 0.1% ethanol (control), 0.2% citrate, 10 μM antimycin A dissolved in 0.1% ethanol, or with 0.2% citrate and 10 μM antimycin A dissolved in 0.1% ethanol. All P. aeruginosa were cultured in rich (PS:DB) medium. *p<0.05, **p<0.01, ***p<0.001.

FIG. 6. NAD(H) ratios and concentrations and fluorescence lifetime g-values of planktonic P. aeruginosa cultured using single carbon sources. (A) NADH/NAD+ ratios, (B) total NADH and NAD+ concentrations, (C) individual NADH and NAD+ concentrations, and (D) fluorescence lifetime phasor values of planktonic wild-type P. aeruginosa that were cultured using single carbon sources. (D) Fluorescence lifetime phasor plot (left) and corresponding g-values (right). Points on the map represent the average fluorescence lifetime of approximately five cells. The centers and axis lengths of the ellipses correspond to the mean and standard deviation, respectively, of all cells pooled from three independent experiments. The arrow indicates the direction towards the position of the free NADH reference, which does not appear on this plot. The bars and errors bars in the bar graph indicate the means and standard deviations of the means of three independent experiments, respectively. The dashed line indicates a metabolic trajectory that connects the average phasor positions for different carbon sources. (E) Total NAD(H) concentrations and fluorescence lifetime g-values plotted together. The best-fit line is indicated with R=−0.86. The points and errors bars indicate the means and standard deviations of the means of three independent experiments, respectively. Planktonic P. aeruginosa were cultured in modified minimal medium and harvested at mid-exponential phase at an optical density OD600 of 0.2 with the exception of the Δphz1/2 strain, which was cultured in modified MOPS medium and harvested at 1 and 4 hours following dilution. *p<0.05.

FIG. 7. Fluorescence lifetimes of pyocyanin and pyoverdine mutants, of purified pyocyanin and pyoverdine molecules, and of wild-type cells treated with antimycin A. The fluorescence lifetime phasor plots (left) and corresponding g values (right) of planktonic (A) pyocyanin (Δphz1/2) mutants or (B) pyoverdine (pvdA−) mutants cultured using 0.2% citrate or glucose as the sole carbon source. (C) Phasor plot indicating the fluorescence lifetimes of purified pyoverdine and pyocyanin. The dashed line indicates the metabolic trajectories of surface-attached (red) and planktonic (blue) P. aeruginosa observed in FIGS. 2E and 6D, respectively. (D) Phasor plot (left) and corresponding g values (right) of planktonic wild-type P. aeruginosa cultured in 0.2% citrate as the sole carbon source and treated with 0.1% ethanol (control) or 10 μM antimycin A dissolved in 0.1% ethanol. Points on the phasor maps represent the average fluorescence lifetime of approximately five cells. The centers and axis lengths of the ellipses correspond to the mean and standard deviation, respectively, of all cells pooled from three independent experiments. The bars and errors bars in the bar graph indicate the means and standard deviations of the means of three independent experiments, respectively. Planktonic P. aeruginosa were cultured in modified minimal medium and harvested at mid-exponential phase at an optical density OD600 of 0.2. *p<0.05, ***p<0.001.

FIG. 8. Host killing indexes and surface densities of wild-type, quorum sensing-defective and surface sensing-defective P. aeruginosa, and fluorescence lifetime g-values and NADH and NAD+ concentrations of planktonic and surface-attached P. aeruginosa during the growth transition. (A) Host killing indexes of surface-attached wild-type, ΔlasR, or ΔpilY1 P. aeruginosa that were assayed for virulence after 0 to 6 hours of growth following dilution from a saturated culture. Host-killing indexes were determined using amoebae as host cells. Bars indicate the average of three independent experiments and errors bars indicate standard deviation. (B) Corresponding surface densities of wild-type, ΔlasR, or ΔpilY1 P. aeruginosa after 6 hours of growth following dilution from a saturated culture. Bars indicate the average of three surface density measurements and errors bars indicate standard deviation. (C) The fluorescence lifetime g-values of surface-attached (red) or planktonic (blue) wild-type, ΔlasR, or ΔpilY1 P. aeruginosa at 4, 5, or 6 hours following dilution from an overnight culture, using the same data set shown in FIG. 4A. Bars and error bars indicate the means and standard deviations, respectively, of the means of three independent experiments. NADH and NAD+ concentrations of (D) surface-attached or (E) planktonic P. aeruginosa during the same period. Bars and error bars indicate the mean and standard deviation of three independent experiments, respectively. P. aeruginosa were cultured in PS:DB medium. *p<0.05, ***p<0.001.

FIG. 9. Impact of supplementing carbon sources on NAD(H) concentrations and surface density in surface-attached P. aeruginosa. (A) NADH and NAD+ concentrations in surface-attached wild-type P. aeruginosa that have been supplemented with the indicated carbon sources at 0.2% concentration at 3 hours following dilution from an overnight culture and cultured for an additional hour. P. aeruginosa were cultured in PS:DB. Bars indicate the average of three experiments and error bars indicate standard deviation. (B) Surface densities of P. aeruginosa from virulence assays after 6 hours of growth. Bars indicate the average of three surface density measurements and errors bars indicate standard deviation.

FIG. 10. Clustering analysis of P. aeruginosa fluorescence lifetimes identifies distinct metabolic states. (A) Five distinct metabolic clusters (C1 to C5) were identified using a K-means clustering algorithm using composite fluorescence lifetime data of P. aeruginosa cells from the current study and from a previous study (37). (B) Silhouette analysis on K-means clustering was used to identify clusters. (C) The cluster score was highest for 5 clusters.

FIG. 11. Schematic summarizing fluorescence lifetime analysis and the host-killing assay. (A) Planktonic or surface-attached P. aeruginosa cells are isolated from the same culture grown in a petri dish, immobilized using an agar pad, and imaged using a fluorescence lifetime microscope. (B) Individual cells or cell clusters are segmented using fluorescence intensity images and an intensity threshold. Fluorescence lifetimes in individual cells or cell clusters are transformed to a 2-dimensional phasor plot containing s and g axes. (C) Planktonic or surface-attached P. aeruginosa cells are isolated from the same culture grown in a petri dish, mixed with axenically-grown amoeba host cells, immobilized with an agar pad containing calcein-AM, and imaged using a fluorescence microscope.

FIG. 12. The FLIM-AST device couples single-cell imaging, which are present in the cutting-edge platforms QuantMatrix and Accelerate Phenosystem, with a divergent AST technology fluorescence lifetime imaging (FLIM) to offer unprecedented level of sensitivity and turnaround time in the assessment of antibiotic susceptibility.

FIG. 13. (A) FLIM-phasors of E. coli that have been exposed to ampicillin. Each data point is the FLIM profile of a single cell within a population. (B) The average values along the g-axis vary with concentration. (C) After drug is removed, the phasor profile recovers to smaller values of g.

FIG. 14. (A) Shift in FLIM g-value following a 1 hour treatment of a tetracycline-sensitive strain of E. coli. (B) Residual residue (RR) values for a range of tetracycline concentrations for a tetracycline-sensitive and tetracycline-resistant strain of E. coli.

### DETAILED DESCRIPTION OF THE INVENTION

### Definitions

All scientific and technical terms used in this application have meanings commonly used in the art unless otherwise specified. As used in this application, the following words or phrases have the meanings specified.

As used herein, “virulent bacteria” refers to bacteria whose growth is harmful or toxic to a subject, such as a human subject.

As used herein, “a” or “an” means at least one, unless clearly indicated otherwise.

As used herein, to “prevent” or “protect against” a condition or disease means to hinder, reduce or delay the onset or progression of the condition or disease.

### Methods

The invention provides methods for inhibiting bacterial virulence by activating metabolic pathways using a carbon source that produces a small g value. In one embodiment, the bacterium is a gram-negative bacterium, such as, for example, Pseudomonas. Examples of Pseudomonas include P. aeruginosa, P. oryzihabitans, and P. plecoglossicida. Another representative example of a gram-negative bacterium is E. coli. In another embodiment, the bacterium is a gram-positive bacterium, such as, for example, S. aureus. The methods described herein can be used to inhibit bacterial virulence on a surface, and/or to inhibit bacterial virulence in a subject suffering from, or at risk of suffering from, a bacterial infection.

In some embodiments, the surface is a medical device or instrument, or other piece of medical or hospital equipment. In one embodiment, the device is a tube or catheter. In other embodiments, the surface is an object that is otherwise brought into contact with the body, such as a contact lens.

In some embodiments, the subject is a hospital or surgical patient, or a person working in a hospital or surgical environment. In some embodiments, the subject is a patient who has or has had contact with a medical device, such as a respirator, tube, or catheter. In some embodiments, the subject has a wound resulting from a burn or from surgery. In some embodiments, the subject is immunocompromised, suffers from diabetes or cystic fibrosis, or is infected with HIV.

In one embodiment, the small or low g value is less than the value of cells at the virulent state. Examples of small or low g values include values of about 0.3. In one embodiment, the method comprises administering a carbon source to a treatment site. In one embodiment, the carbon source is one that produces a low g value, for example, relative to a free NADH reference. Representative carbon sources for use in the methods include, but are not limited to, pyruvate and citrate. Additional examples of carbon sources include oxaloacetate, malate, and fumarate.

The carbon source can be administered to the treatment site by various modes of delivery, including, but not limited to, topical application, injection into a site, and systemic delivery. The mode of delivery will be selected by the treating physician based on the needs of the subject to be treated.

For use in the methods described herein, representative examples of the treatment site include, but are not limited to, a burn wound, an incision, or other site that is infected or at risk of infection.

### Compositions

The invention provides compositions for use in inhibiting bacterial virulence. Such compositions comprise a carbon source as described herein and, optionally, a pharmaceutically acceptable carrier or excipient.

The composition is formulated in accordance with the mode of administration. For example, the composition may be a gel, paste, cream, aqueous, or other formulation, as appropriate for topical application, direct application to a wound or incision, or systemic delivery.

### Device, System, Computer-Readable Media

Also provided is a device for testing antibiotic susceptibility of bacteria. In some embodiments, the device comprises (a) a receiving surface adapted to receive and immobilize bacteria in contact with a test antibiotic; (b) a fluorescence lifetime imaging microscopy (FLIM) apparatus that emits an excitation pulse of light directed at the receiving surface; (c) a detector that collects time-correlated fluorescence emitted from individual bacteria immobilized on the receiving surface; (d) an analyzer that correlates time-correlated fluorescence emitted from individual bacteria collected by the detector with the excitation pulse emitted by the FLIM apparatus to generate a FLIM-phasor profile; and (e) an analyzer that correlates the FLIM-phasor profile generated in step (d) to the status of the antibiotic susceptibility of the bacteria. In some embodiments, the detector collects fluorescence with nanosecond resolution. The device can be used in a method of testing antibiotic susceptibility of bacteria isolated from a patient sample, wherein the method comprises immobilizing bacteria isolated from a patient sample onto the receiving surface of the device. In one embodiment, the method comprises: (a) immobilizing bacteria isolated from a patient sample onto a receiving surface; (b) measuring the FLIM signatures at an initial time point upon contacting the immobilized bacteria with a test antibiotic and at a plurality of intervals for 30 minutes to 1 hour; (c) directing a series of nanosecond excitation pulses of light at the immobilized bacteria; (d) collecting time-correlated fluorescence emitted from individual bacteria immobilized on the receiving surface; (e) generating FLIM-phasor profiles by taking the sine and cosine transform of the fluorescence intensity decays, thereby generating s and g values; and (f) comparing FLIM-phasor profiles obtained before and after the contacting of step (b); wherein a change in the g value upon contact with a test antibiotic indicates bacterial susceptibility to the test antibiotic. In some embodiments, the test antibiotic is selected from the group consisting of amoxicillin (penicillin-type), cephalexin (cephalosporin), erythromycin (macrolide), ciprofloxacin (fluoroquinolone), trimethoprim (sulfonamide), tetracycline, and gentamicin (aminoglycoside). In some embodiments, steps (c)-(e) of the method are repeated at intervals of 10-20 minutes for 1-3 hours after contacting the bacteria with test antibiotic. In some embodiments, steps (c)-(e) of the method are repeated at intervals of 15 minutes for 2 hours after contacting the bacteria with test antibiotic. In some embodiments, the method is completed in less than one hour.

Also provided is a system for testing antibiotic susceptibility of bacteria, the system comprising a user device comprising a hardware processor that is programmed to generate and analyze FLIM-phasor profiles as recited above. Representative examples of antibiotics include, but are not limited to, tetracycline, ampicillin, carbenicillin, chloramphenicol, spectinomycin, gentamycin, and kanamycin. Additionally provided is a non-transitory computer-readable medium containing computer executable instructions that, when executed by a processor, cause the processor to generate and analyze FLIM-phasor profiles as recited above.

### EXAMPLES

The following examples are presented to illustrate the present invention and to assist one of ordinary skill in making and using the same. The examples are not intended in any way to otherwise limit the scope of the invention.

### Example 1: Shift in Central Metabolism Underlies Virulence Induction in Pseudomonas aeruginosa

Details of this Example can be found in Perinbam K, et al., 2020, mBio 11:e02730-18 (doi.org/10.1128/mBio.02730-18). The availability of energy has significant impacts on cell physiology. However, the role of cellular metabolism in bacterial pathogenesis has not been understood. This Example investigates the dynamics of central metabolism during virulence induction by surface sensing and quorum sensing in early-stage biofilms of the multi-drug resistant bacterium Pseudomonas aeruginosa. We established a metabolic profile for P. aeruginosa using fluorescence lifetime imaging microscopy (FLIM), which reports the activity of nicotinamide adenine dinucleotide (NADH) in live cells. We identified a critical growth transition period during which virulence is activated. We performed FLIM measurements and direct measurements of NADH and NAD+ concentrations during this period. Here, planktonic (low-virulence) and surface-attached (virulence-activated) populations diverged into distinct metabolic states, with the surface-attached population exhibiting FLIM lifetimes that were associated with lower levels of enzyme-bound NADH and decreasing total NAD(H) production. We inhibited virulence by perturbing central metabolism using citrate and pyruvate, which further decreased the enzyme-bound NADH fraction and total NAD(H) production, and suggested the involvement of the glyoxylate pathway in virulence activation in surface-attached populations. In addition, we induced virulence at an earlier time using the electron transport chain oxidase inhibitor antimycin A. Our results demonstrate the use of FLIM to non-invasively measure NADH dynamics in biofilms and suggest a model in which a metabolic rearrangement accompanies the virulence activation period.

The rise of antibiotic resistance requires the development of new strategies to combat bacterial infection and pathogenesis. A major direction has been the development of drugs that broadly target virulence. However, few targets have been identified due to the species-specific nature of many virulence regulators. The lack of a virulence regulator that is conserved across species has presented a further challenge to the development of therapeutics. In this Example, we identify that NADH activity has an important role in the induction of virulence in the pathogen P. aeruginosa. This finding, coupled with the ubiquity of NADH in bacterial pathogens, opens up the possibility for targeting enzymes that process NADH as a potential broad anti-virulence approach.

Pseudomonas aeruginosa is an opportunistic pathogen that is responsible for a range of illnesses including lung infection in cystic fibrosis patients, hospital-acquired infections, sepsis, and disease in immunocompromised patients (1). The bacterium infects a broad range of hosts including humans, animals, plants, insects, amoebae, and other bacteria using a multitude of virulence factors including type III secretion, cyanide, pyocyanin, and proteases (2-5). Recent work has reported that the expression of virulence factors in P. aeruginosa is regulated by nutrient availability and central metabolic networks (6-8). In addition, tricarboxylic acid cycle (TCA) intermediates alter the activity of some virulence factor regulators in Gram-positive and intracellular pathogens (9, 10). These studies provide a static snapshot of the involvement of metabolism in virulence regulation. The dynamics of central metabolic activity during the activation of virulence in P. aeruginosa are not known and many questions remain about the regulatory link between central metabolism and virulence activation. Unknown are the energetic requirements for the expression of virulence factors, or whether central metabolism can be tuned to inhibit virulence. This Example addresses these questions by measuring central metabolic activity during the transition from a low-virulence state to an activated virulence state in P. aeruginosa.

Virulence factor production is induced in P. aeruginosa and other bacteria through the activation of surface sensing (11-15). The host-killing mechanism of surface-activated virulence in P. aeruginosa has not been attributed to a single virulence factor, including type III secretion, pyocyanin, or elastase, but has been attributed to the combinatorial nature of virulence factor production (2, 12). In addition, a recent pre-print reports that alkyl quinolones are a critical cytotoxic factor (16). Virulence induction by surface attachment is dependent on the protein PilY1, which is found on the outer surface of the cell membrane, contains homology to a mechanically-active von Willebrand factor domain, mediates a c-di-GMP response to shear stress, and is required for the initiation of biofilm formation (17-21). Surface-induced virulence also requires co-activation of quorum sensing, which is triggered when cells reach a threshold density (22, 23). Surface sensing and quorum sensing form a coincidence gate in which the activation of both pathways is required to induce virulence (12).

Surface attachment regulates the levels of the metabolites cyclic AMP and cyclic-di-GMP (13, 20, 21, 24) and upregulates transcription of NADH-associated enzymes (12). Quorum sensing, which controls the expression of many virulence factors, produces a major shift in the production of a large fraction of metabolites (25). It is possible that surface sensing and quorum sensing induce virulence through changes in central metabolism. However, addressing this hypothesis has been challenging because surface sensing and quorum sensing are dynamic processes and monitoring their effects requires simultaneous measurements of both virulence and central metabolism.

The phasor approach to fluorescence lifetime imaging microscopy (FLIM) measures the dynamics of central metabolism in live cells. This method reports the relative abundance of the free and bound forms of NADH by exploiting its autofluorescent properties (26, 27). This method has been used extensively to track changes in NADH forms in live eukaryotic cells during critical cell processes including duplication, proliferation, and differentiation (28-32). NADH is excited using two-photon excitation and decays to the ground state with distinct decay rates, or lifetimes, in the visible spectrum. The major advantage of this approach is the ability to track spatial and temporal changes in metabolic activity at sub-cellular resolution without the need to label molecules, introduce fluorescent reporters, or to stain, perturb, or harvest cells. Advances in optics, imaging, and analysis have enabled fluorescence lifetime measurements in a number of bacteria including L. acidophilus, E. coli, and P. aeruginosa (33-37). However, independent measurements of NADH were not performed in these studies, which limited the interpretation of the FLIM measurements. In addition, the FLIM measurements were not performed during virulence activation.

This Example establishes a metabolic trajectory in P. aeruginosa using FLIM and through independent in vitro measurements of NADH and NAD⁺ concentrations. We measure metabolic states in P. aeruginosa during a critical growth transition in which virulence is activated in surface-attached cells. We show that compared to low-virulence (planktonic) cells, virulence-activated (surface-attached) cells exhibit FLIM lifetimes that are associated with decreased levels of enzyme-bound NADH and decreased NAD(H) production. Perturbation of central metabolism using citrate and pyruvate, which induce decreases in enzyme-bound NADH and total NAD(H) production, inhibits virulence, while treatment using an electron transport chain oxidase inhibitor induces virulence at an earlier time.

Materials and Methods

Growth Conditions and Strains

Pseudomonas aeruginosa strains were streaked onto LB-Miller (BD Biosciences, Franklin Lakes, N.J.) petri dishes containing 2% Bacto agar (BD Biosciences) and incubated at 37° C. to obtain single colonies. Individual colonies were inoculated into modified PS:DB, which is a rich medium that supports co-culturing of P. aeruginosa cells with Dictyostelium discoideum (amoeba) (12). The modified PS:DB medium, hereafter referred to as PS:DB medium in this work, is the same formulation of PS:DB as described previously (12) except that PS medium was used at a concentration of 90% (v/v) instead of 10% (v/v). P. aeruginosa strains were cultured overnight in PS:DB in a rotary drum rotating at 24 rpm or orbital shaker rotating at 200 rpm and 37° C., diluted 1:100 into a plastic or glass dish containing the same medium, and cultured between 4 to 6 hours. Alternatively, strains were cultured in minimal medium A (61) containing 0.2% glucose, diluted 1:100 into minimal media A that was modified to exclude citrate (hereafter referred to as modified minimal medium A) and containing one of the following carbon sources at a concentration of 0.2%: glucose, glycerol, citrate, or pyruvate (Sigma, St. Louis, Mo.), and cultured to an optical density (OD₆₀₀) of 0.2 at 37° C.

D. discoideum was grown axenically in PS medium at 22° C. as described previously (12) and harvested for virulence assays when cultures reached an optical density at 600 nm (OD₆₀₀) between 0.2 to 0.5.

Wild-type P. aeruginosa strain PA14 (62), PA14 strains containing a ΔlasR (AFS20.1) (63) or ΔpilY1 (19) deletion, a pvdA::Mar2xT7 mutation (64), or the ΔphzA1-G1 ΔphzA2-G2 mutations (65, 66) (original strain name DKN330, referred to here as Δphz1/2), and the D. discoideum strain AX3 (67) were used for these experiments.

Fluorescence Lifetime Imaging Microscopy (FLIM)

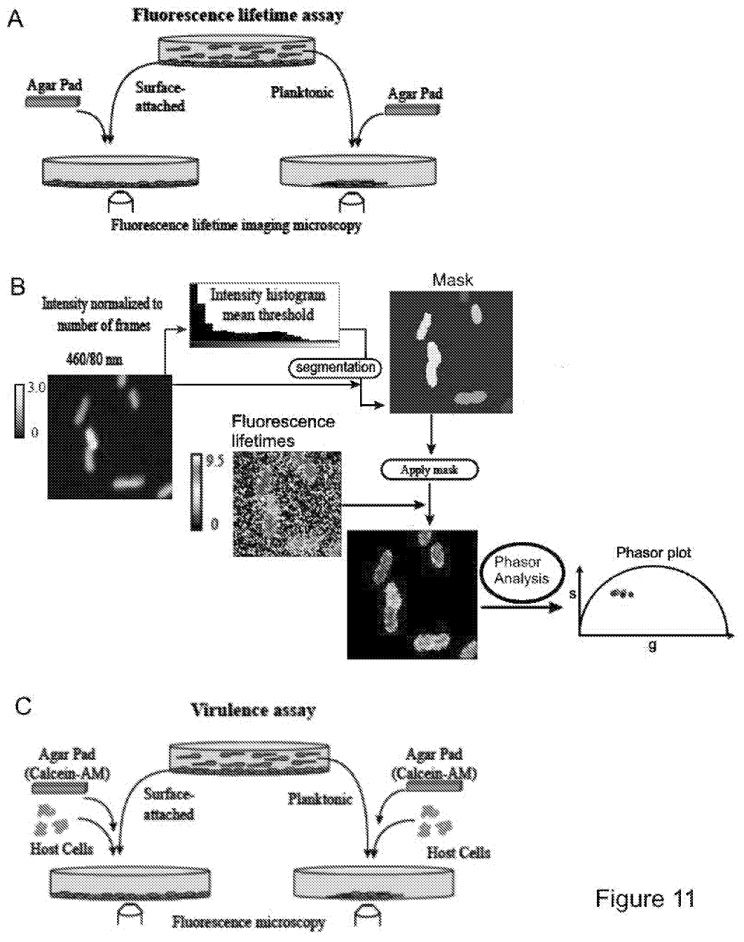

Fluorescence lifetime measurements were performed using a custom-built multiphoton microscope setup based on an Olympus FV1000 system and an Olympus IX81 microscope (Olympus, Waltham, Mass.) as described previously (68). The FLIM microscope uses an 80 MHz ultrafast Ti:Sapphire Mai Tai laser (Spectra-physics, Santa Clara, Calif.) set at 740 nm for mulitphoton excitation. The setup used a 690-nm SP dichroic-460/80 nm filter pair for separating emission and a PlanApo N Olympus oil immersion 60× (1.42 NA) objective (Olympus, Waltham, Mass.), which is capable of bacterial single-cell resolution imaging. An H7422P-40 photomultiplier tube module (Hamamatsu, Bridgewater, N.J.) and A320 FastFLIM Box (ISS, Champaign, Ill.) were used to measure fluorescence lifetime. Image acquisition was controlled by SimFCS software version 4 (64-bit) (Laboratory for Fluorescence Dynamics, Irvine, Calif.). Planktonic and surface-attached cells were isolated by modifying a protocol described previously (12, 50) (FIG. 11A).

Masks for bacterial cells were created through SimFCS using fluorescence intensity images and image intensity thresholding (FIG. 11B). Fluorescence lifetimes within the masked areas were transformed using

$${g}_{i , j} ( ω ) = \text{?} {I}_{i , j} ( t ) cos ( ω t ) dt \text{/} \text{?} {I}_{i , j} ( t ) dt and {s}_{i , j} ( ω ) = \text{?} {I}_{i , j} ( t ) sin ( ω t ) dt \text{/} \text{?} {I}_{i , j} ( t ) dt , \\ \text{?} \text{indicates text missing or illegible when filed}$$

where I(t) is the fluorescence intensity decay, ω is the laser repetition angular frequency, and the indexes i and j identify a pixel of the image.

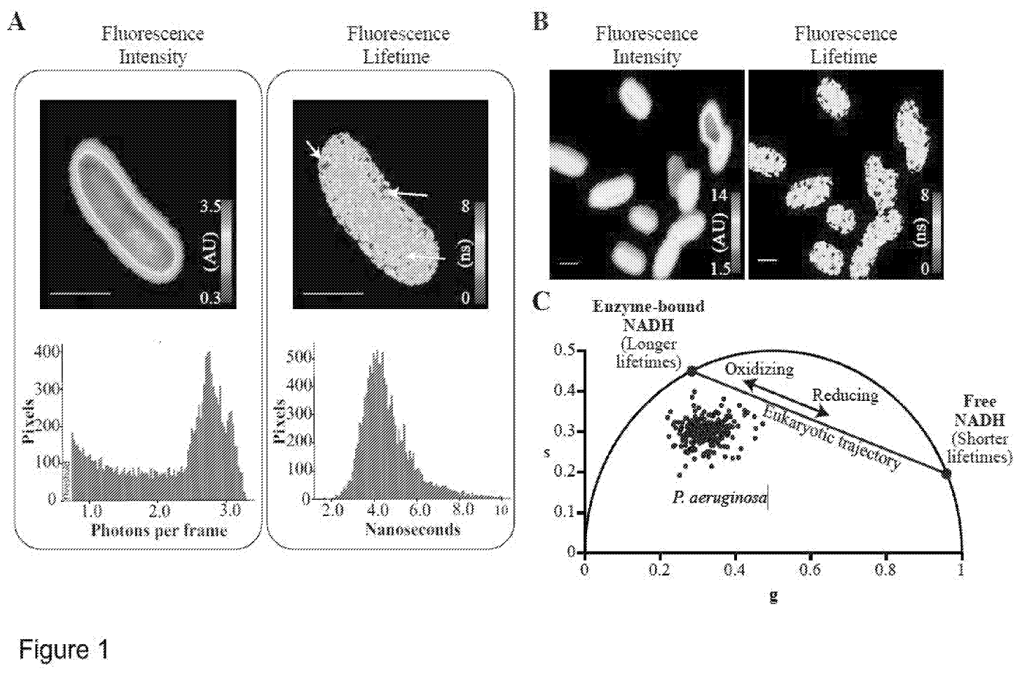

The microscope was calibrated before each session by setting the fluorescence lifetime obtained for 10 μM rhodamine 110 (Sigma, St. Louis, Mo.) to 4.0 ns. The laser power was set to 20% (<3 mW at back aperture of the microscope) using an acousto-optic modulator (AA Opto Electronic, Orsay, France). Sub-cellular fluorescence lifetimes (FIG. 1A) were collected using the 20× digital zoom mode at a rate of 1.7 sec/frame. All other P. aeruginosa measurements were performed using the 6× digital zoom mode using the same frame rate. For all measurements, 40 sequential frames were acquired to generate a single FLIM image.

Virulence Assay

P. aeruginosa cells were assayed using an image-based virulence assay as described previously (12, 50). P. aeruginosa strains were cultured in PS:DB, diluted 1:100 into 60×15 mm plastic petri dishes (Corning, Corning, N.Y.), cultured for 4 to 6 hours by shaking at 100 rpm, and harvested. Planktonic cells were assayed by transferring 10 μL of culture from the petri dish to a new petri dish, mixing with an equal volume of D. discoideum that were grown to an OD₆₀₀ of 0.2-0.5, and immobilizing by placing an agar pad on top of the mixture (FIG. 11C). Agar pads were made using Bacto agar at a concentration of 1%, DB buffer, and 1 μM calcein acetoxymethyl (AM) ester (Molecular Probes, Eugene, Oreg.), and were cut into 1.5×1.5 cm squares (12, 50). Surface-attached cells were assayed by aspirating planktonic cells from the culture, washing with DB buffer to remove planktonic cells, mixing with an equal volume of D. discoideum, and immobilizing by placing an agar pad on top of the mixture (FIG. 11C) (50). The immobilized planktonic or surface-attached P. aeruginosa and D. discoideum were incubated at room temperature for 1 hour and imaged using fluorescence microscopy. The host killing index (12, 50) was computed as the average ratio of calcein-AM fluorescence to cell area in individual amoeba cells for 30 to 200 cells in each experiment.

Fluorescence microscopy to assess calcein-AM fluorescence was performed using a Nikon Eclipse Ti-E microscope (Nikon, Melville, N.Y.) containing Nikon 10× Plan Fluor Ph1 (0.3 NA) and 20×S Plan Fluor Nikon (0.45 NA) objectives, a Sola light engine (Lumencor, Beaverton, Oreg.), an LED-DA/FI/TX filter set (Semrock, Rochester, N.Y.) containing a 409/493/596 dichroic and 474/27 nm and 525/45 nm filters for excitation and emission, respectively, and a Hamamatsu Orca Flash 4.0 V2 camera (Hamamatsu, Bridgewater, N.J.). Images were acquired using Nikon NIS-Elements and analyzed using custom built software written previously (12) in Matlab (Mathworks, Natick, Mass.).

Nicotinamide Adenine Dinucleotide (NADH, NAD⁺) Concentrations

The concentrations of reduced and oxidized nicotinamide adenine dinucleotide (NADH, NAD⁺) were measured using a colorimetric enzyme-cycling assay as described previously (45). P. aeruginosa strains were cultured overnight in PS:DB, diluted 1:100 into 60×15 mm plastic petri dishes (Corning, Corning, N.Y.) containing the same medium, and grown for 4 to 6 hours. Alternatively, strains were cultured overnight in minimal medium, diluted 1:100 into plastic petri dishes containing modified minimal medium with single carbon sources, and grown to an OD₆₀₀ of 0.2. Cultures were harvested in a glove box chamber (Bel-Art, Wayne, N.J.) that had been vacuumed to remove air and flushed with nitrogen constantly throughout the experiment to prevent oxidation of NADH. Subsequent steps were performed and solutions were prepared in the glove box unless otherwise indicated. Solutions containing 0.05 to 1 μM of NADH (Sigma) or NAD⁺ (Sigma) were included as calibration controls.

Planktonic cells were isolated from petri dishes, pelleted by centrifugation at 16,000×g for 1 min, the residual supernatant was discarded and the pellet was immediately resuspended in 0.2 M NaOH or HCl to extract NADH or NAD⁺, respectively, as described previously (45, 46). Surface-attached cells were harvested by aspirating planktonic cells from petri dishes, washing with DB buffer or modified minimal medium with no carbon source for strains cultured in PS:DB or modified minimal medium A containing single carbon sources, respectively, adding 0.2 M NaOH or HCl, and scraping surfaces with a cell scraper (Sarstedt, Nümbrecht, Germany). As a control, the Δphz12 strain was cultured overnight in modified MOPS synthetic medium (46), diluted 1:100 in the same medium in culture tubes for 1 to 4 hours, pelleted by centrifugation, resuspended in 0.2 M NaOH or HCl, as described previously (46). The resuspensions in NaOH or HCl were neutralized using equal volume of 0.1 M HCl or NaOH, respectively, and portions were assayed for protein content, NADH, and NAD⁺. Resuspensions were assayed for protein content using a BCA assay kit (ThermoFisher Scientific, Waltham, Mass.) using a 50:1 mixture of solutions A and B from the kit. Solutions were incubated at 37° C. for 30 min and measured for absorbance at 562 nm. Resuspensions that were not used for the BCA assay were normalized by protein content by diluting in Millipore-filtered water.

NADH and NAD⁺ concentrations were determined by following the protocol described previously (45). Briefly, normalized resuspensions were centrifuged at 16,000×g for 10 minutes at 4° C. to remove cell debris and mixed with a reaction mixture containing 40 mM EDTA, 1 M bicine (Sigma), 4.2 mM 3-(4,5-dimethylthiazol-2-yl)-2,5-diphenyltetrazolium bromide (Sigma), 16.6 mM phenazine ethosulfate (Sigma), 1 mg/mL alcohol dehydrogenase (Sigma), and ethanol, and the absorbance at 570 nm of the solution was measured in a 96-well plate (Corning Corning, N.Y.) at 30° C. every 30 seconds for 30 minutes using a BioTek Synergy HTX reader (BioTek, Winooski, Vt.). The reaction mixture and the normalized resuspensions were aliquoted and mixed inside the glove box. The centrifugation and absorbance measurements were performed outside of the glove box. The absorbance per unit time was determined by fitting data on the initial velocity of enzyme activity, acquired between 2 to 10 minutes of the absorbance measurements. The absorbances per unit time for the NADH and NAD⁺ calibration controls were linearly fit for the full range of calibration concentrations to establish a linear equation that relates the absorbance per unit time and NAD(H) concentration. The NAD(H) concentrations of the planktonic and surface-attached cell resuspensions were determined by inputting the respective absorbance per unit time fits of the samples into these equations.

Statistical Analysis

The significance of changes between experimental conditions was determined by using unpaired two-tailed heteroscedastic t-tests. The t values, degrees of freedom, and p values are given in Table 1. For fluorescence lifetime phasor plots, the center of ellipses indicates the mean of all cells pooled from multiple experiments. The axis lengths of the ellipses indicate the standard deviation of the pooled data set. Unless otherwise indicated, bars and error bars in bar graphs indicate the mean and standard deviation of the means from multiple independent experiments, respectively.

TABLE 1

| | | | Degrees of | |
|---|---|---|---|---|
| FIG. | Comparison | t value | freedom | p value |
| 2B | Citrate, Glycerol | 2.56 | 2 | 1.25E−01 |
| 4A | Surface-attached, planktonic (WT, | 8.17 | 2 | 1.47E−02 |
| | 5 hrs) | | | |
| 4A | Surface-attached, planktonic (WT, | 25.70 | 3 | 1.29E−04 |
| | 6 hrs) | | | |
| 4B | Surface-attached, planktonic (WT, | 23.93 | 4 | 1.81E−05 |
| | 5 hrs) | | | |
| 4B | Surface-attached, planktonic (WT, | 12.06 | 4 | 2.71E−04 |
| | 6 hrs) | | | |
| 4C | Surface-attached, planktonic (WT, | 12.62 | 2 | 6.22E−03 |
| | 5 hrs) | | | |
| 4C | Surface-attached, planktonic (WT, | 11.13 | 2 | 7.98E−03 |
| | 6 hrs) | | | |

Further details on growth conditions, measurements of growth profiles, FLIM measurements, surface density measurements, and the classifier model are described in the Supplemental Information available at Perinbam K, et al., 2020, mBio 11:e02730-18 (doi.org/10.1128/mBio.02730-18).

Results

Fluorescence Lifetimes Shift with Changes in NAD(H) Production in P. aeruginosa

We measured the fluorescence intensities and lifetimes of unlabeled wild-type P. aeruginosa using two-photon microscopy with excitation at 740 nm and emission centered at 460 nm with an 80 nm bandwidth (NADH emission spectrum). Studies in eukaryotic cells have established that fluorescence intensity is proportional to the concentration of intracellular NADH or NADPH and fluorescence lifetime is determined by the relative fraction of NADH or NADPH that is free or bound to proteins (28-31, 38-41). The fluorescence lifetimes of NADH and NADPH are indistinguishable using the techniques in this study and thus we do not differentiate between these species and refer to NADH only. We cultured P. aeruginosa in minimal medium and measured fluorescence at mid-exponential phase. Fluorescence intensities were uniformly distributed in the cytoplasmic region of the cell and dropped off sharply at the periphery (FIG. 1A-B). In contrast, fluorescence lifetimes were relatively heterogeneous within the cytoplasm and longer lifetimes were localized to multiple clusters within the cell (FIG. 1A-B).

We analyzed fluorescence lifetimes using the phasor approach, which determines the relative fraction of free and enzyme-bound forms of NADH and is insensitive to the total intracellular concentration of NADH (26, 27). In this approach, fluorescence decay curves are transformed by cosine and sine functions and plotted along g and s axes, respectively. Fluorescence lifetimes that arise from a single species map to the ‘universal circle’ of the phasor (26, 27) (black semi-circle in FIG. 1C). Free (unbound) NADH has a relatively short lifetime (0.4 ns) and maps to a coordinate on the lower-right position of the universal circle (FIG. 1C). NADH molecules that are bound to eukaryotic enzymes have longer lifetimes (3.2 to 9 ns) and map to coordinates on the upper-left region of the universal circle (28, 29, 39, 40) (FIG. 1C). The positions of free and enzyme-bound NADH on the universal circle serve as reference points. Lifetime decay curves that are generated by a mixture of free and enzyme-bound NADH species, which is typically observed in fluorescence lifetime measurements that are averaged over entire eukaryotic cells, map along a linear trajectory that connects the two reference points on the universal circle (28, 29, 40) (FIG. 1C).

Previous studies in eukaryotic cells demonstrate that growth in oxidizing conditions, which decreases the relative free NADH pool and the ratio of free to enzyme-bound NADH, results in increased lifetimes and phasor positions that are further away from the free NADH reference point (28-30) (FIG. 1C). Conversely, growth in a reducing environment or blocking oxidative phosphorylation through metabolic inhibitors, both which increase the relative free NADH pool, results in decreased lifetimes and phasor positions that are closer to the free NADH reference (28-30) (FIG. 1C). These studies establish that the position of lifetimes along the linear trajectory is an indicator of the relative abundance of free and enzyme-bound NADH within the cell.

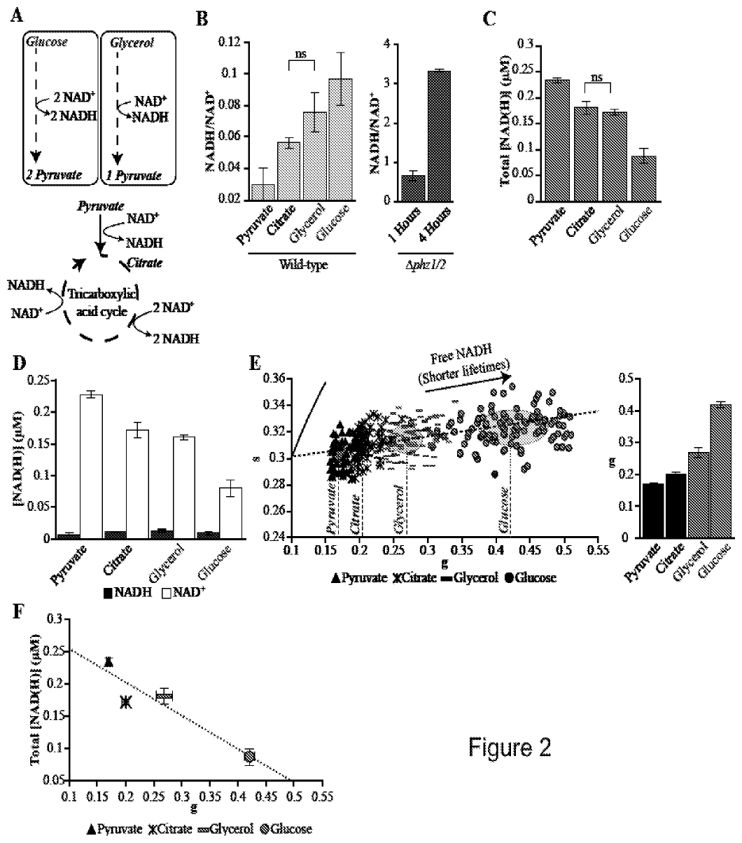

In our experiments, P. aeruginosa cells mapped outside of the eukaryotic NADH trajectory (FIG. 1C). The central metabolic enzymes that bind NADH in P. aeruginosa and eukaryotes are distinct (42, 43) and thus there is no expectation that lifetimes for eukaryotes and bacteria should appear along the same trajectory. To establish a metabolic trajectory for P. aeruginosa, we cultured cells in minimal media supplemented with single carbon sources that are associated with different levels of NADH production. Citrate is metabolized through the TCA cycle, which reduces NAD⁺ to NADH (FIG. 2A). Glycerol and glucose are metabolized through the Entner-Doudoroff pathway, which produces NADH molecules and pyruvate, the latter of which can enter the TCA cycle (42-44). We expected that the metabolism of citrate, glycerol, glucose, or pyruvate produces different levels of NADH (FIG. 2A).

We quantified the concentrations of intracellular NADH and NAD⁺ in surface-attached cells that were cultured in each of the carbon sources (FIG. 2B-D) using an enzyme cycling-based colorimetric assay (45, 46). This assay serves as an independent approach to measuring changes in NADH in addition to the FLIM technique. We observed that the assay was sensitive to the concentration of P. aeruginosa and therefore normalized samples by total protein concentration. We included measurements of a Δphz12 strain, which does not produce pyocyanin, to identify the protein concentration required to measure changes in NADH and NAD⁺. The NADH/NAD⁺ ratio of this strain increases significantly during the transition from early-exponential to late-exponential phase (46). The observed increase in ratio between these growth phases (FIG. 2B) established that the P. aeruginosa concentrations in our samples were within the dynamic range of the assay.

Consistent with expectations, we observed that each carbon source yielded a different NADH/NAD⁺ ratio (FIG. 2B). In particular, pyruvate produced the smallest ratio whereas citrate, glycerol, and glucose yielded increasing ratios. The total concentrations of NADH and NAD⁺ were highest for pyruvate, intermediate for citrate and glycerol, and smallest for glucose (FIG. 2C). The changes in the NADH/NAD⁺ ratios and total concentrations of NAD(H) were primarily due to changes in NAD⁺ production, as the NADH concentrations were relatively constant (FIG. 2D).

We hypothesized that the changes in NADH/NAD⁺ ratios and NAD(H) concentrations are accompanied by changes in the relative fractions of enzyme-bound and free NADH, which would be observed as changes in fluorescence lifetime. We measured fluorescence lifetimes of surface-attached P. aeruginosa at single-cell resolution that were cultured under the same conditions as the NAD(H) measurements. We observed significant changes in fluorescence lifetimes primarily along the g-axis (FIG. 2E). Growth using pyruvate yielded the smallest g-value whereas growth using citrate, glycerol and glucose yielded increasing g-values (FIG. 2E). Interestingly, g-values anti-correlated with total NAD(H) concentrations (FIG. 2F, R=−0.94). To our knowledge, this relationship has not been identified previously in P. aeruginosa.

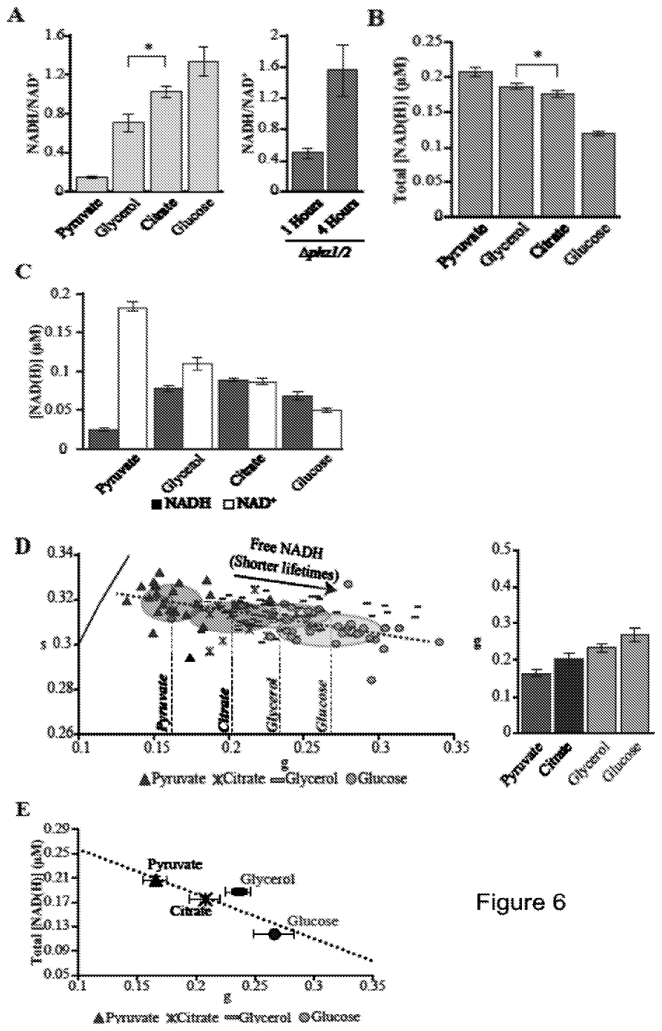

We repeated NAD(H) and fluorescence lifetime measurements using planktonic cells that were isolated from cell pellets from cultures as described previously (45, 46). The trends in NADH/NAD⁺ ratios and total NAD(H) concentrations for these cells (FIG. 6A-C) were identical to that of surface-attached cells (FIG. 2B-D) with the exception that in planktonic cells, citrate produced a slightly greater NADH/NAD⁺ ratio and slightly lower NAD(H) concentration than glycerol (FIG. 6A-B). However, the differences in NADH/NAD⁺ ratios and total NAD(H) concentrations using citrate or glycerol as a carbon source in surface-attached cells were not significant (FIG. 2B-C). The trend in fluorescence lifetime g-values in planktonic cells (FIG. 6D) was identical to that of surface-attached cells (FIG. 2E). Fluorescence lifetime g-values and total NAD(H) concentrations in planktonic cells were also anti-correlated (FIG. 6E, R=−0.86). These results indicate that the impacts of carbon sources on FLIM and NAD(H) production were qualitatively comparable for both planktonic and surface-attached P. aeruginosa. We note that the changes in NAD(H) concentrations in planktonic cells arose from changes in both NADH and NAD⁺ (FIG. 6C), in contrast to surface-attached cells, where changes only in NAD⁺ were observed (FIG. 2D).

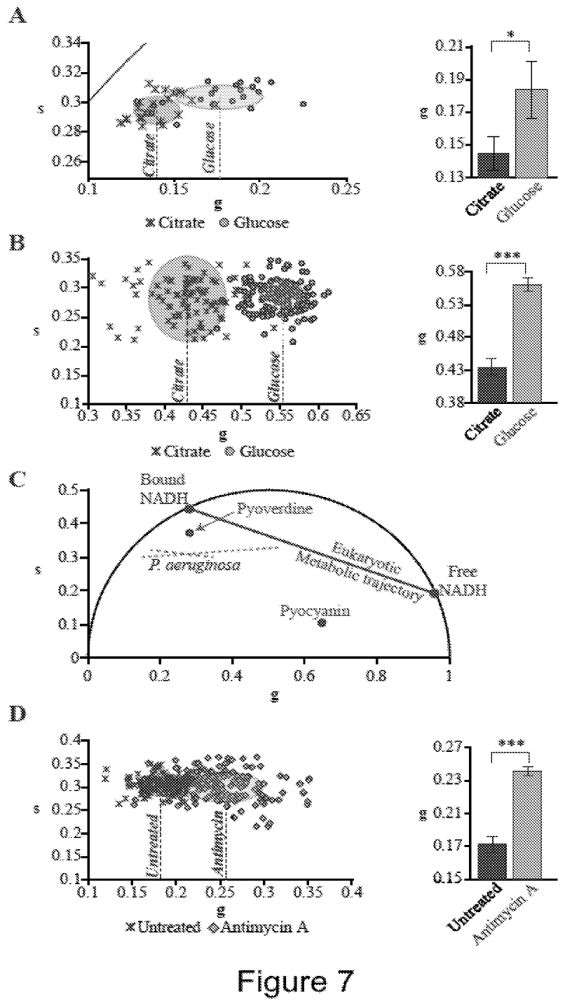

The relative shifts in fluorescence lifetime are not due to pyoverdine or pyocyanin, which are fluorescent molecules produced in high abundance by P. aeruginosa (46, 47), because the lifetime shifts were observed in strains that are defective in the production of pyoverdine or pyocyanin (FIG. 7A-B). Purified forms of pyoverdine and pyocyanin also mapped to positions outside of the P. aeruginosa lifetime range (FIG. 7C). Furthermore, a shift towards higher g-value (greater fraction of free NADH) was observed when P. aeruginosa was treated with the oxidase inhibitor antimycin A, which inhibits electron transport chain activity (48, 49) (FIG. 7D). Together, these results establish a fluorescence lifetime metabolic activity trajectory for P. aeruginosa (FIGS. 2E and 6D). In particular, the P. aeruginosa trajectory is positioned below the eukaryotic metabolic trajectory (FIG. 7C) on the phasor diagram and the shifts are mostly along the g-axis. These data indicate that the selection of carbon source affects the total NAD(H) production and the relative fractions of free and enzyme-bound NADH. The observation that changes in NAD(H) production are anti-correlated with changes in fluorescence lifetime (FIGS. 2F and 6E) suggest that NAD(H) production is tied with the binding of NADH to enzymes. In particular, lower NAD(H) production is associated with a lower fraction of enzyme-bound NADH (higher g-values) whereas a shift towards higher NAD(H) production is associated with a higher fraction of enzyme-bound NADH (lower g-values). While a strong correspondence between fluorescence lifetimes and NADH activity is observed here, we note that it is possible that other autofluorescent molecules produced by P. aeruginosa contribute to the fluorescence lifetime measurements. The results here suggest that NADH is the predominant determinant of fluorescence lifetime in our experiments.

A Growth Transition Accompanies Virulence Induction

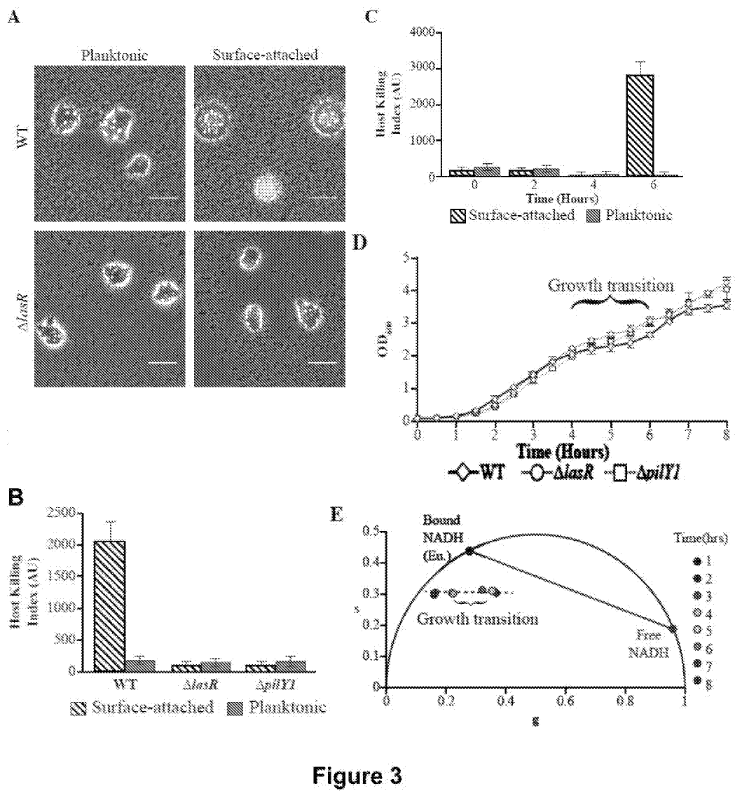

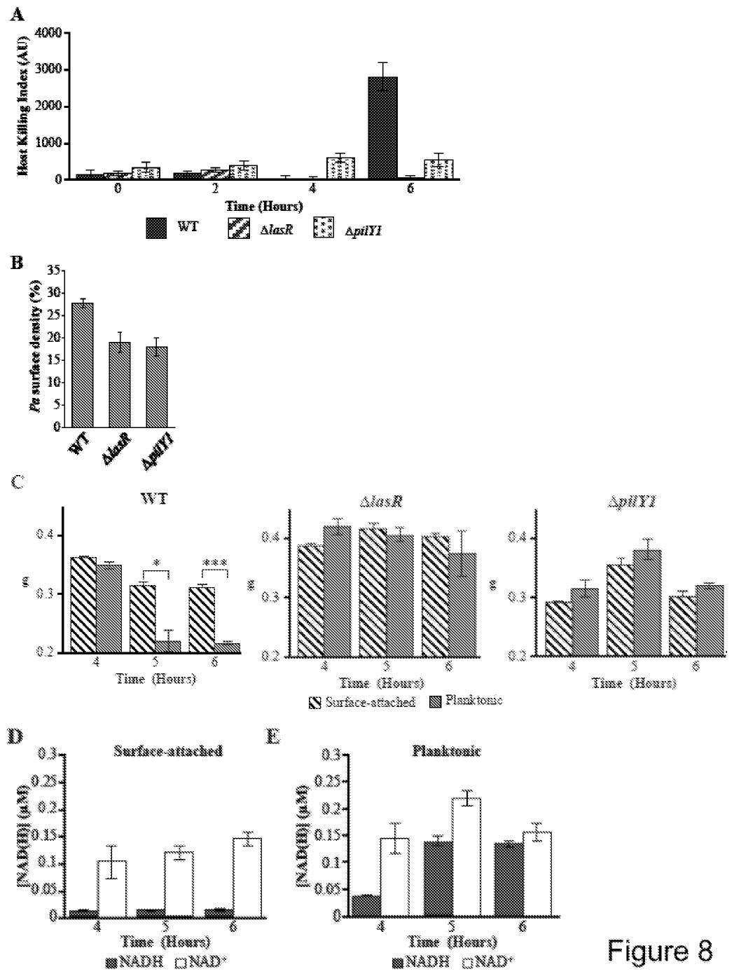

We characterized changes in fluorescence lifetime during the onset of virulence. Virulence was measured using an image-based assay using amoebae as host cells (12, 50). This assay is sensitive to virulence-activated and low virulence phenotypes but does not capture intermediate virulence phenotypes (50). P. aeruginosa that are attached to a rigid substrate transition from a low-virulence state to a virulence-activated state during late-exponential phase (12, 13). We confirmed that surface-attached P. aeruginosa cells activate virulence in our growth conditions in rich medium using amoebae as host cells and a calcein-AM stain, which fluoresces when amoebae are stressed (12, 50) (FIG. 3A-B). We also confirmed that surface-activated virulence requires the master quorum sensing regulator LasR and the surface sensing-associated protein PilY1 (12, 21) (FIGS. 3A-B and 8A). The mutations decrease the density of P. aeruginosa on the surface (FIG. 8B). However, we do not attribute the changes in virulence to the decrease in surface density, as the roles of LasR and PilY1 in regulating surface-activated virulence have been established previously (12).

To identify the growth period during which virulence is induced, we measured virulence at 2-hour intervals following dilution from an overnight saturated culture. Virulence was induced in surface-attached cells during a critical period between 4 to 6 hours of growth (FIG. 3C). Interestingly, this critical period was marked by a growth transition that punctuated two distinct periods of exponential growth, as measured by the optical density of planktonic cells from cultures in flasks (FIG. 3D). The growth transition is consistent with a diauxic shift in which the cellular growth rate is temporarily reduced while the enzymes required for utilizing a different metabolic pathway are produced (51). Growth transitions were observed in LasR and PilY1 mutants, which have low virulence, although the transition effects were significantly diminished in these strains (FIG. 3D). To determine whether central metabolic activity is altered during the growth transition, we measured the fluorescence lifetimes of planktonic cells from cultures in flasks. We observed a significant shift in fluorescence lifetimes between 4-5 hours, coinciding with the growth transition, towards smaller values of g (FIG. 3E). These results suggest that P. aeruginosa undergoes a significant metabolic rearrangement during the virulence induction period.

Virulence-Activated and Low-Virulence Populations are Metabolically Distinct

Planktonic and surface-attached populations develop distinct virulence phenotypes during the growth transition period. At the start of the transition period at 4 hours, both populations are in a low-virulence state (FIG. 3C-D). By the end of the transition period, surface-attached cells are induced for virulence whereas planktonic cells remain in the low-virulence state (FIG. 3C-D). We hypothesized that planktonic and surface-attached cells undergo distinct metabolic changes during the growth transition period.

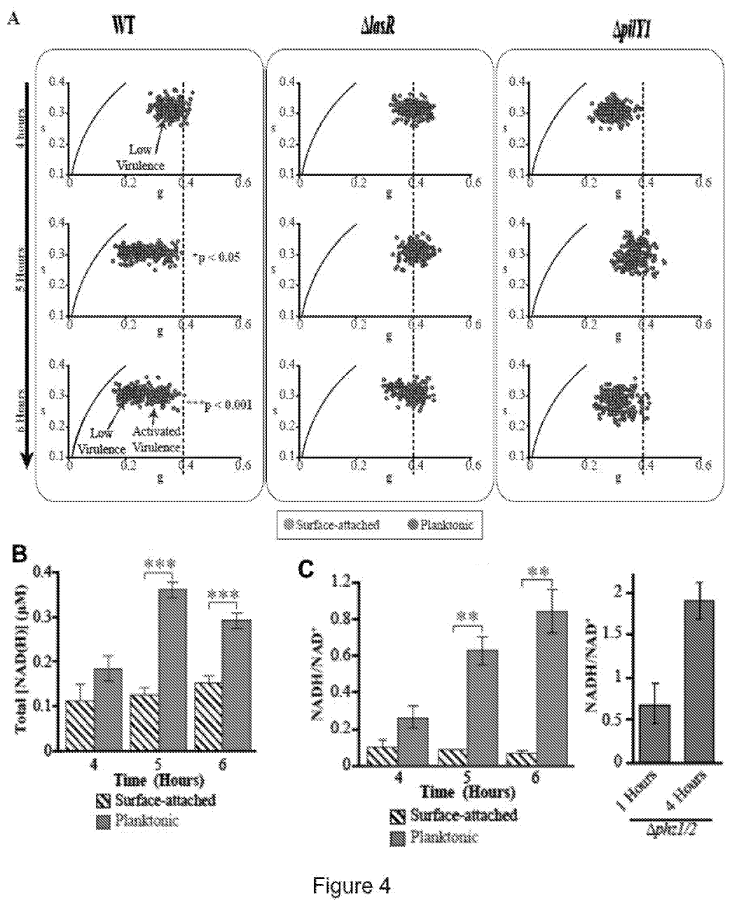

We monitored the fluorescence lifetimes and NAD(H) production in both populations from the same culture during the growth transition between 4-6 hours. At the start of the growth transition (4 hrs following inoculation), the fluorescence lifetime profiles of the two populations were indistinguishable and had comparable g-values (FIGS. 4A, upper-left and 8C). Two hours following entry into the growth transition (6 hrs following inoculation), in which P. aeruginosa enters a new period of exponential growth (FIG. 3D), the two populations exhibited distinct metabolic profiles (FIGS. 4A bottom-left and 8C). Surface-attached cells shifted to a smaller g-value of 0.3 whereas planktonic cells shifted towards a significantly smaller g-value of 0.2 (FIGS. 4A, bottom-left, and 8C). Measurements using the enzyme-cycling assay indicated that the sub-populations produced distinct concentrations of NAD(H) after two hours (FIG. 4B), with the surface-attached population producing significantly lower levels of total NAD(H). This effect was due primarily to lower levels of NADH in the surface-attached population (FIG. 8D-E). Consequently, the NADH/NAD⁺ ratios in surface-attached P. aeruginosa were significantly lower than those in the planktonic population (FIG. 4C).

These results indicate that surface-attached and planktonic populations have distinct g-values, total NAD(H) concentrations, and NADH/NAD⁺ ratios towards the end of the growth transition. In surface-attached P. aeruginosa, the decreased NAD(H) production, lower NADH/NAD⁺ ratios and greater abundance of free NADH, as indicated by the FLIM measurements, are consistent with a less active metabolic state compared to the planktonic population. These results suggest that planktonic and surface-attached P. aeruginosa enter the growth transition period with identical metabolic states but exit the period with distinct metabolic activities, with the planktonic population having greater NADH-associated metabolic activity than the surface-attached population.

Surface-attached and planktonic cells have distinct host-killing activities at the end of the growth transition, with surface-attached and planktonic cells exhibiting virulence-activated or low-virulence states, respectively (FIG. 3C). Coupled with the observation that surface-attached and planktonic populations have distinct metabolic states, these observations suggest a link between metabolic states and virulence (FIG. 4A). We investigated whether metabolic activity is altered in LasR and PilY1 mutants, which have significantly reduced host-killing activity (12, 19-21, 15, 16). Surface-attached or planktonic populations of these mutants do not kill amoebae (FIGS. 3A-B and 8A). We found that the FLIM g-values of both surface-attached and planktonic populations of these mutants were indistinguishable during the growth transition (FIG. 4A). In particular, we note the higher baseline g-value of 0.4 in the LasR mutant, which suggests a higher abundance of free NADH in this strain. The LasR mutant is impaired in the production of the pyocyanin (52), which functions as a major electron acceptor and reacts with NADH (46). The shift to a higher g-value in the LasR mutant may reflect a lack of interaction between NADH and pyocyanin.

Discussion

Bacterial virulence is regulated by a number of factors that ensure successful infection. How the metabolic state of the cell changes during virulence induction has been unknown. Our results indicate that a shift in central metabolism, in the form of changes in NADH and NAD⁺ abundances and NADH binding to enzymes, accompanies the induction of virulence in P. aeruginosa. Using this finding, we perturb central metabolism to inhibit virulence or to induce virulence at an earlier time. As NADH is utilized as a central metabolic currency broadly across bacterial species, our results suggesting a role for NADH abundance in the regulation of virulence could have far-reaching significance.

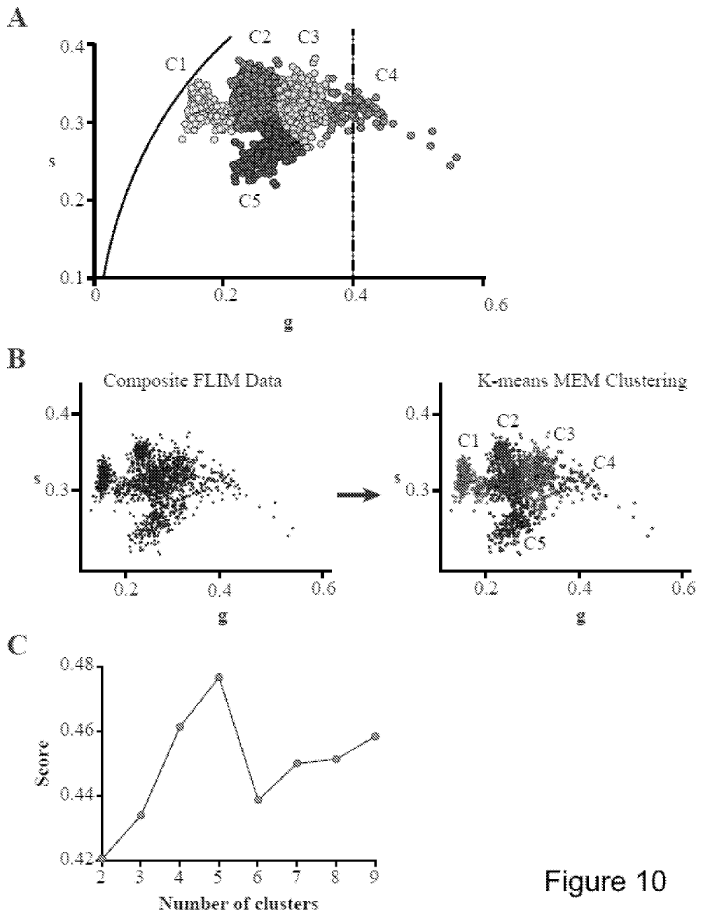

We have established a metabolic trajectory in P. aeruginosa using the phasor approach to fluorescence lifetime imaging microscopy. We observed that positions along the g-axis of the fluorescence lifetime trajectory negatively correlated with total NAD(H) production. Greater FLIM g-values, indicating decreased enzyme-bound NADH within the cell, correlated with decreases in NAD(H) production. In addition, analysis of the cumulative fluorescence lifetime data using a K-means entropy clustering algorithm identified 5 distinct metabolic states into which P. aeruginosa cells can be clustered (FIG. 10).

By establishing fluorescence lifetime maps and performing NAD(H) concentration measurements and host killing assays, we have measured the dynamics of central metabolism in P. aeruginosa during the activation of virulence. Our analysis revealed that P. aeruginosa undergoes a rapid and distinct metabolic rearrangement during the growth transition that differentiates cells into low-virulence or virulence-activated populations. At the beginning of the growth transition when P. aeruginosa entered a period of reduced growth rate, both planktonic and surface-attached populations were metabolically indistinguishable by FLIM and NAD(H) measurements. At the end of the transition, planktonic populations had an increased proportion of enzyme-bound NADH and increased the production of NAD(H) but did not activate host-killing factors. In contrast, surface-attached populations had relatively decreased enzyme-bound NADH and decreased NAD(H) production, which resembled a state of metabolic dormancy, and transitioned to an activated virulence state.

Virulence is observed in our experiments only in surface-attached cells. Low-virulence planktonic cells produce greater levels of NAD(H) and have greater enzyme-bound NADH. The mechanisms that give rise to the distinct metabolic states is unclear. The availability of electron acceptors and surface sensing in P. aeruginosa could have an impact on metabolism. These results thus suggest an important role for electron transfer activity in the activation of virulence mechanisms. Future experiments will need to address the extent to which NAD(H) production and free NADH in planktonic cells impact the production of host-killing factors.

Fluorescence lifetime imaging microscopy provides spatial measurements of metabolism and may be a useful tool for measuring metabolic activity across multiple length scales from single-cells to mature biofilms. We observed that fluorescence lifetimes were spatially heterogeneous in the cytoplasm of P. aeruginosa, which is consistent with the sub-cellular localization of metabolic activity (58). Future experiments will need to address the impact of changes in central metabolism on the spatial organization of NADH activity. In addition, metabolic dormancy in biofilms is associated with antibiotic resistance (59). The use of FLIM to map spatial changes in metabolism in biofilms may thus open new avenues for the investigation of antibiotic resistance in biofilms.

Anti-virulence therapy is a proposed strategy for combatting pathogenesis as an alternative to conventional antibiotics, which typically target bacterial growth (60). The identification that NADH levels affect virulence induction highlights a potential target for virulence inhibition. Our results suggest metabolic manipulation as a strategy to inhibit virulence. Strategies such as targeting metabolic pathways involved in NAD(H) production or growth in the presence of bacteria that secrete metabolites that affect NAD(H) production, could be effective at inhibiting virulence. Within microbiomes, complex microbial communities, and host environments, metabolite cross-feeding could have a significant impact on virulence activation in pathogens.

### REFERENCES

1. Driscoll J A, et al. 2007. Drugs 67:351-368.
2. Lee D G, et al. 2006. Genome Biol 7:R90.
3. Rahme L G, et al. 2000. Proc Natl Acad Sci USA 97:8815-8821.
4. Pukatzki S, et al. 2002. Proc Natl Acad Sci USA 99:3159-3164.
5. Hauser A R. 2009. Nat Rev Microbiol 7:654-665.
6. Van Alst N E, et al. 2007. Infect Immun 75:3780-3790.
7. Palmer G C, Whiteley M. 2015. Microbiol Spectr 3. doi.org/10.1128/microbiolspec.MBP-0003-2014.
8. Bartell J A, et al. 2017. Nat Commun 8:14631.
9. Eisenreich W, et al. 2010. Nat Rev Microbiol 8:401-412.
10. Somerville G A, Proctor R A. 2009. Microbiol Mol Biol Rev 73:233-248.
11. Gode-Potratz C J, et al. 2011. Mol Microbiol 79:240-263.
12. Siryapom A, et al. 2014. Proc Natl Acad Sci 111:16860-16865.
13. Persat A, et al. 2015. Proc Natl Acad Sci 112:7563-7568.
14. Alsharif G, et al. 2015. Proc Natl Acad Sci 112:5503-5508.
15. Chuang S K, et al. 2019. Nat Commun 10:4118.
16. Vrla G D, et al. 2019. Microbiology doi.org/10.1101/773291.
17. Bohn Y-S T, et al. 2009. Mol Microbiol 71:730-747.
18. Heiniger R W, et al. 2010. Cell Microbiol 12:1158-1173.
19. Kuchma S L, et al. 2010. J Bacteriol 192:2950-2964.
20. Rodesney C A, et al. 2017. Proc Natl Acad Sci 114:5906-5911.
21. Luo Y, et al. 2015. mBio 6:e02456-14.
22. Pesci E C, et al. 1997. J Bacteriol 179:3127-3132.
23. Miller M B, Bassler B L. 2001. Annu Rev Microbiol 55:165-199.
24. O'Connor J R, et al. 2012. Mol Microbiol 86:720-729.
25. Davenport P W, et al. 2015. J Bacteriol 197:2072-2082.
26. Clayton A H A, et al. 2004. J Microsc 213:1-5.
27. Digman M A, et al. 2008. Biophys J 94:L14-16.
28. Stringari C, et al. 2012. Sci Rep 2:568.
29. Wright B K, et al. 2012. Biophys J 103:L7-L9.
30. Stringari C, et al. 2012. PLoS ONE 7:e48014.
31. Quinn K P, et al. 2013. Sci Rep 3:3432.
32. Stringari C, et al. 2015. Cell Rep 10:1-7.
33. Jakobs S, et al. 2000. FEBS Lett 479:131-135.
34. Micic M, et al. 2004. Colloids Surf B Biointerfaces 34:205-212.
35. Gee M L, et al. 2013. Sci Rep 3. doi.org/10.1038/srep01557.
36. Torno K, et al. 2013. Curr Microbiol 66:365-367.
37. Bhattacharjee A, et al. 2017. Sci Rep 7. doi.org/10.1038/s41598-017-04032-w.
38. Bird D K, et al. 2005. Cancer Res 65:8766-8773.
39. Stringari C, et al. 2011. Proc Natl Acad Sci 108:13582-13587.
40. Wright B K, et al. 2012. Microsc Res Tech 75:1717-1722.
41. Mayevsky A, Rogatsky G G. 2007. Am J Physiol-Cell Physiol 292:C615-C640.
42. Lessie T G, Phibbs P V. 1984. Annu Rev Microbiol 38:359-388.
43. Conway T. 1992. FEMS Microbiol Lett 103:1-28.
44. Williams H D, et al. 2006. Oxygen, Cyanide and Energy Generation in the Cystic Fibrosis Pathogen Pseudomonas aeruginosa, p. 1-71. In Advances in Microbial Physiology. Elsevier 52:1-71.
45. Kern S E, et al. 2014. Extraction and Measurement of NAD(P)+ and NAD(P)H, p. 311-323. In Filloux, A, Ramos, J-L (eds.), Pseudomonas Methods and Protocols. Springer New York, N.Y., N.Y. 1149:311-323.
46. Price-Whelan A, et al. 2007. J Bacteriol 189:6372-6381.
47. Meyer J-M. 2000. Arch Microbiol 174:135-142.
48. Matsushita K, et al. 1980. J Bacteriol 141:389-392.
49. Shvinka J E, et al. 1979. J Gen Microbiol 113:377-382.
50. Perinbam K, Siryapom A. 2018. J Vis Exp doi.org/10.3791/57844.
51. Monod J. 1949. Annu Rev Microbiol 3:371-394.
52. Lau G W, et al. 2004. Trends Mol Med 10:599-606.
53. Lindsey T L, et al. 2008. Microbiol Read Engl 154:1616-1627.
54. Chung J C S, et al. 2013. Open Biol 3:120131.
55. Crousilles A, et al. 2018. J Biol Chem 293:14260-14269.
56. Vrla G D, et al. 2019. Microbiology doi.org/10.1101/773291.
57. Farrow J M, Pesci E C. 2007. J Bacteriol 189:3425-3433.
58. Ingerson-Mahar M, et al. 2010. Nat Cell Biol 12:739-746.
59. Meylan S, et al. 2017. Cell Chem Biol 24:195-206.
60. Rasko D A, Sperandio V. 2010. Nat Rev Drug Discov 9:117-128.
61. Miller J H. 1992. Cold Spring Harbor Laboratory Press, Plainview, N.Y.
62. Rahme L G, et al. 1995. Science 268:1899-1902.
63. O'Loughlin C T, et al. 2013. Proc Natl Acad Sci 110:17981-17986.
64. Liberati N T, et al. 2006. Proc Natl Acad Sci 103:2833-2838.
65. Dietrich L E P, et al. 2006. Mol Microbiol 61:1308-1321.
66. Ramos I, et al. 2010. Res Microbiol 161:187-191.
67. Loomis W F. 1971. Exp Cell Res 64:484-486.
68. Trinh A, et al. 2017. Cancers 9:168.

### Example 2: Use of Carbon Sources to Inhibit Virulence in Pseudomonas aeruginosa

This Example describes new methods of inhibiting bacterial virulence by the counterintuitive method of activating metabolic pathways using a carbon source having a low g value. Although one would expect that supplying energy would activate virulence, the data in Example 1 show that providing a carbon source that produces a low g value actually inhibits virulence in Pseudomonas aeruginosa. Thus, we describe here the use of pyruvate, citrate, and other carbon sources that produce a low g value, to inhibit bacterial virulence.

The inhibition of bacterial virulence can be achieved by exposing a site or surface that may be contaminated with Pseudomonas aeruginosa to pyruvate, citrate, or another carbon sources that produces a low g value. The exposure can be achieved by directly contacting a surface with the carbon source, or by applying a medicament or other composition containing the carbon source to a site on or within a subject's body. For example, the composition can be applied to a wound, into a surgical incision, or at site internal or external to the body at which Pseudomonas aeruginosa is present.

Growth Conditions

Citric acid or pyruvic acid (Sigma, St. Louis, Mo.) were used as carbon sources in experiments involving measurements of NAD(H) concentrations. Sodium citrate or sodium pyruvate were used in experiments involving FLIM measurements of planktonic cells, except for FIG. 7D, for in which citric acid was used. Planktonic and surface-attached cells were isolated by modifying a protocol described previously (1, 2).

Fluorescence Lifetime Imaging Microscopy Conditions

P. aeruginosa strains were cultured overnight in PS:DB, diluted 1:100 into 35 mm glass-bottom dishes (MatTek Corporation, Ashland, Mass.) containing the same medium, and cultured for 4 to 6 hours (FIGS. 2E-F, 4A, 5A, and 8C). Alternatively, strains were cultured overnight in minimal medium A, diluted 1:100 into modified minimal media A with single carbon sources in culture tubes (FIGS. 6D-E and 7A-B, D) or glass-bottom dishes and cultured to an OD₆₀₀ of 0.2 (FIG. 2E-F).

Planktonic cells were transferred to a new glass-bottom dish, immobilized using an agar pad, and imaged immediately (FIG. 11A). Agar pads were made using 1% Bacto agar (BD, Franklin Lakes, N.J.) and DB buffer (1, 2) or modified minimal medium A containing no carbon source for strains that were cultured in PS:DB or modified minimal medium, respectively, and were cut into 1.5×1.5 cm squares. Surface-attached cells were isolated by aspirating the supernatant from the dish, washing the dish to remove planktonic cells with DB buffer or modified minimal medium with no carbon source for strains cultured in PS:DB or modified minimal medium, respectively, and placing an agar pad on the dish surface, and were imaged immediately. Imaging was performed at room temperature.

For experiments involving FLIM measurements of the effects of antimycin A (FIG. 7D), P. aeruginosa were grown in culture tubes to saturation in modified minimal medium A containing 0.2% citrate, diluted 1:100 into the same medium in culture tubes, supplemented with 10 μM antimycin A (Sigma, St. Louis, Mo.) with 0.1% ethanol or with 0.1% ethanol, cultured to an OD₆₀₀ of 0.2, and immediately measured for fluorescence lifetime. Fluorescence lifetimes for pyocyanin and pyoverdine (FIG. 7C) were measured using solutions of 6.2 mM pyocyanin (P0046, Sigma, St. Louis, Mo.) in DMSO and 5 mg/mL pyoverdine (P8124, Sigma, St. Louis, Mo.) in deionized water, respectively. For FIG. 6D, cells with fluorescence intensities below a value of 1.0, as reported by the SimFCS software, were excluded from the analysis.

Measurement of Growth Profiles

The growth profiles of P. aeruginosa (FIG. 3D) were measured by diluting saturated overnight cultures to 1:100 in PS:DB into 50 mL of the same medium in a 250 mL Erlenmeyer flask, culturing at 37° C. with shaking at 200 rpm, and measuring the OD₆₀₀ of 1 mL of the culture every 30 mins using an Ultrospec10 spectrometer (Harvard Bioscience Inc., Holliston, Mass.). Cultures with OD₆₀₀ measurements greater than 0.5 were diluted into fresh PS:DB and re-measured. The final OD₆₀₀ was computed by normalizing the measurement by the dilution factor.

Supplementation with Carbon Sources or Antimycin a to Surface-Attached Cells

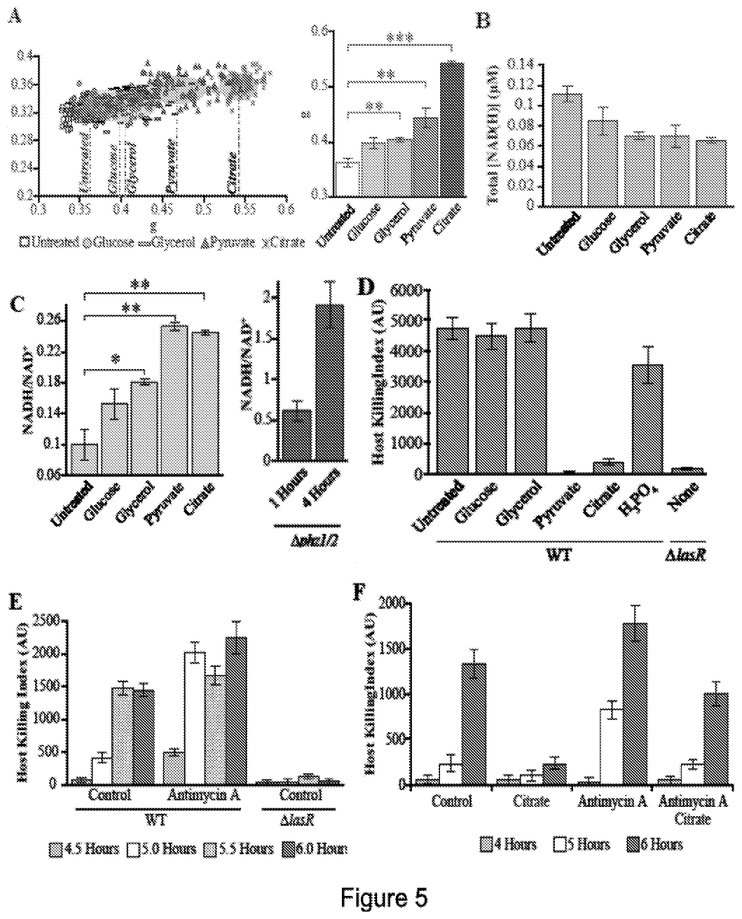

For experiments in which strains cultured in PS:DB were supplemented with individual carbon sources (FIG. 5A-D), pyruvic acid, citric acid, phosphoric acid, glycerol, or glucose was added to cultures at a final concentration of 0.2% at 3 hours following a 1:100 dilution from an overnight culture. Cultures were harvested after 1 additional hour of growth for measurements of fluorescence lifetime and NADH and NAD⁺ concentrations. For experiments in which surface-attached strains that were cultured in PS:DB media were treated with antimycin A (FIG. 5E-F), 10 μM antimycin A dissolved in 0.1% ethanol, 0.1% ethanol, alone, or 10 μM antimycin A dissolved in 0.1% ethanol and 0.2% citric acid were added after 3 hours of growth following a 1:100 dilution from an overnight culture. Host killing was measured after an additional 1 to 3 hours of growth (total of 4 to 6 hours of growth).

Surface Density Measurements

The density of P. aeruginosa cells on surface was measured as described previously (1) using amoebae cell viability phase contrast images that were acquired using a 10× or 20× objective. The IJ_Isodata algorithm (ImageJ 1.52q) was applied to phase contrast images to construct cell boundary masks. The cell density was computed by dividing the area covered by P. aeruginosa by the total area of the image.

Classifier Model

K-means clustering was performed using the Scikit kmeans classifier (3) and minimum cluster entropy was estimated by maximizing the silhouette coefficient score (4) (FIG. 10).

Perturbation of Central Metabolism Alters Virulence Activation

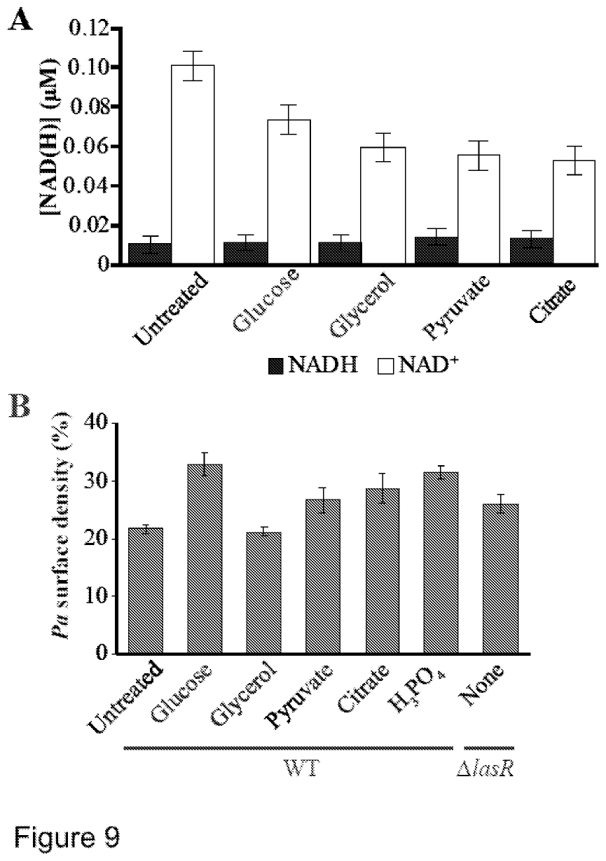

We investigated the extent that altered central metabolic activity affects host-killing activity in surface-attached cells. We cultured P. aeruginosa in rich medium and supplemented the cultures at the beginning of growth transition with carbon sources that mapped to distinct positions along the fluorescence lifetime trajectory (FIG. 2E) and that produced distinct changes in NAD(H) production (FIG. 2C). We observed that all carbon sources increased the FLIM g-values, decreased NAD(H) production, and increased NADH/NAD⁺ ratios (FIG. 5A-C). The changes in NAD(H) production and NADH/NAD⁺ ratios were due primarily to decreases in NAD⁺ production (FIG. 9A). No change in NADH production was detected. Citrate and pyruvate produced the greatest increases in g-values, consistent with significant decreases in the relative abundance of enzyme-bound NADH, caused the greatest decreases in NAD(H) production, and caused the greatest increases in NADH/NAD⁺ ratio (FIG. 5A-C). In contrast, supplementation with glucose induced relatively small changes in FLIM g-value, NAD(H) production, and NADH/NAD⁺ ratio (FIG. 5A-C). These results suggest that the treatment of surface-attached P. aeruginosa with carbon sources at the beginning of the growth transition induces the populations into metabolic states that have different levels of NADH activity.

Analysis of host-killing activity revealed that citrate inhibited virulence in surface-attached P. aeruginosa (FIG. 5D). Similar results were observed using pyruvate, which had similar impacts on FLIM g-values, NAD(H) production, and NADH/NAD⁺ ratios as citrate (FIG. 5A-C). In contrast, treatment with glucose or glycerol, which produced the smallest changes in NADH/NAD⁺ ratios and FLIM g-values, had no effect on host-killing activity. The reduction in host-killing activity by citrate or pyruvate was not due to changes in the density of P. aeruginosa on surfaces, as the treatments did not decrease the surface density (FIG. 9B). The reduction in host-killing activity was also not due to changes in pH, as treatment with phosphoric acid at the same concentration had little impact on host-killing activity (FIG. 5D). These results suggest that a change in NAD(H) activity and enzyme-bound NADH fraction inhibits the activation of host-killing factors.

In separate experiments, we supplemented cultures at the beginning of the growth transition with antimycin A, which inhibits NADH oxidation. Treatment of surface-attached P. aeruginosa with antimycin A induced virulence 30 minutes earlier than untreated cells (FIG. 5E). This effect was inhibited by the co-treatment of antimycin A with citrate (FIG. 5F). Table 2 provides t values, degrees of freedom, and p values for unpaired two-tailed heteroscedastic t-tests that appear in the figures.

TABLE 2

| | | | Degrees of | |
|---|---|---|---|---|
| FIG. | Comparison | t value | freedom | p value |
| 5A | Untreated, glycerol | 8.08 | 3 | 3.96E−03 |
| 5A | Untreated, pyruvate | 6.29 | 3 | 8.12E−03 |
| 5A | Untreated, citrate | 36.86 | 3 | 4.39E−05 |
| 5C | Untreated, glycerol | 7.29 | 2 | 1.83E−02 |
| 5C | Untreated, pyruvate | 13.51 | 2 | 5.43E−03 |
| 5C | Untreated, citrate | 13.01 | 2 | 5.86E−03 |
| 6A | Glycerol, citrate | 4.97 | 3 | 1.56E−02 |
| 6B | Glycerol, citrate | 3.08 | 4 | 3.69E−02 |
| 7A | Citrate, glucose | 3.38 | 3 | 4.30E−02 |
| 7B | Citrate, glucose | 14.92 | 4 | 1.18E−04 |
| 7D | Untreated, antimycin A | 14.11 | 3 | 7.71E−04 |
| 8C | Surface-attached, planktonic (WT, | 8.17 | 2 | 1.47E−02 |
| | 5 hrs) | | | |
| 8C | Surface-attached, planktonic (WT, | 25.70 | 3 | 1.29E−04 |
| | 6 hrs) | | | |

The observation that virulent (surface-attached) populations are metabolically distinct from low-virulence (planktonic) populations raises the possibility that altering central metabolism activity could affect virulence activation. Treatment of surface-attached P. aeruginosa with citrate and pyruvate decreased the enzyme-bound NADH pool, decreased the total NAD(H) production, and abolished host-killing activity. In contrast, glucose and glycerol had relatively small impacts on the level of enzyme-bound NADH and NAD(H) production, and had no effect on host-killing activity.

The impacts of individual carbon sources on host-killing activity may be interpreted in the context of the glyoxylate pathway, which bypasses the TCA cycle in favor of carbon preservation for gluconeogenesis and biomass production. The glyoxylate pathway activates the expression of type Ill secretion and is important for lung infection models (53, 54). Growth in citrate and pyruvate in P. aeruginosa biases metabolic activity in favor of the TCA pathway and away from the glyoxylate pathway (55). Thus, the inhibition of virulence observed here could be explained by the inhibition of the glyoxylate pathway by citrate or pyruvate. Treatment of surface-attached P. aeruginosa using an oxidase inhibitor induced virulence at an earlier time, which was also inhibited by treatment of citrate. Glucose does not appear to inhibit the glyoxylate pathway and glycerol is not expected to inhibit the pathway (55). Consistent with our interpretation, supplementation with glucose or glycerol had no impact on host-killing activity. Together, our results suggest a model in which the glyoxylate pathway is activated in surface-attached populations, which results in the expression of host-killing factors. In this model, the activation of the pathway can be inhibited by citrate or pyruvate but not glucose or glycerol. The observed changes in NAD(H) production and the fraction of enzyme-bound NADH may be indicative of changes in TCA and glyoxylate pathway utilization. The decreased production of NAD(H) in surface-attached populations is consistent with inactivation of the TCA cycle in favor of the glyoxylate pathway.

A recent pre-print indicates that alkyl quinolines are responsible for cytotoxicity in surface-associated populations (56). Anthranilate is a metabolic precursor for quinolones (57) and its availability may have a significant impact on the production of these cytotoxic factors in surface-associated populations. The alteration of central metabolites could thus function as a regulator to rapidly coordinate virulence during the critical growth transition period.

### REFERENCES

1. Siryaporn A, et al. 2014. Proc Natl Acad Sci 111:16860-16865.
2. Perinbam K, Siryaporn A. 2018. J Vis Exp doi.org/10.3791/57844.
3. Pedregosa, F., et al. 2011. JMLR 12:2825-2830.
4. Rousseeuw P J. 1987. J Comput Appl Math 20:53-65.

### Example 3: Fluorescence Lifetime Imaging Microscopy Device for Antibiotic Susceptibility Testing (FLIM-AST)

This Example describes development of a FLIM-AST device for the rapid determination of the antibiotic susceptibility of bacteria. This device can determine antibiotic susceptibility within 2 hours after bacteria have been isolated from patient samples. The FLIM-AST device determines antibiotic susceptibility by measuring bacterial metabolism in single bacterial cells. The FLIM-AST device measures antibiotic susceptibility using single-microscopy and fluorescence lifetime imaging microscopy (FLIM), which is a method that does not require bacterial growth. This device takes advantage of the single-cell microscopy and couples this with a non-destructive measurement of metabolic activity through FLIM. In addition, the FLIM-AST device can work in all bacteria, as the central metabolic molecules are common in all known bacteria

Antibiotics have had a significant impact on society by extending lifespans and treating bacterial infections that could otherwise lead to fatalities of millions of people (1-4). However, bacterial strains that are resistant to a large number of antibiotics have rapidly emerged in healthcare settings globally (3,5). In 2017, the WHO identified that the Gram-negative strains Pseudomonas aeruginosa, Acinetobacter baumannii, and Enterobacteriaceae, pose the most serious threats (6).

In healthcare settings, patients are commonly infected by bacteria that are resistant (not susceptible) to antibiotics. In order for medical care providers to prescribe effective treatments, it is essential to determine whether patients are infected by antibiotic-resistant strains and if so, which antibiotics are effective at treating the infection. Currently, the technology to distinguish between resistant and non-resistant strains requires between 4 to 12 hours and a relatively large volumes of blood (10-20 mL) or patient specimen.

The invention here decreases the time required for diagnosis, with a target of assessment within 2 hours. The information delivered by the FLIM-AST device will enable doctors to determine: (1) whether the current course of antibiotic treatments is effective at eradicating the infection, and (2) identifying potential antibiotics that are more effective at treating the infection. Use of this device could cut down on the critical wait time towards treatment of bacterial infections and enable personalized medical treatments, which will improve patient treatment outcomes and decrease the rise of antibiotic resistance.

A number of devices are currently offered on the market that assess antibiotic resistance. The important features to consider when evaluating these devices are: (1) time to antibiotic susceptibility assessment, (2) whether assessment can be made using patient samples, and (3) whether the assessment is based either on a functional response to antibiotics or on DNA. An ideal AST device would make the assessment within 30 minutes directly from patient samples and would be based on a functional response. There is currently no device on the market that meets all 3 of these criteria.

Most devices on the market rely on the measurements of growth (7), which requires 4-16 hours due to the requirement for bacteria to replicate from ˜10 CFU/mL and due to limitations of sensitivity. During this critical wait period, patient health can drop precipitously. Many products are available to perform growth-based measurements, for example the VITEK 2 AST card, which requires 4-13 hours for assessment. Coupling bacterial growth with higher sensitivity instruments offers a potential decrease in detection time. For example, the Bacterioscan FLLS combines growth with high intensity laser scanning for optical density measurements and can provide assessments within 6 hours directly from samples. In addition, the BD Phoenix system combines redox indicators, decreasing the time of assessment to 4 hours.

Single-cell microscopy has been implemented as a method to enhance bacterial detection sensitivity as an alternative to bulk measurements. The Accelerate Phenosystem uses single-cell fluorescence microscopy and fluorescence in-situ hybridization (FISH) of probes to detect and identify bacteria directly from patient samples within 7 hours. The QuantaMatrix System (8,9) provides an assessment within 3 hours and relies on single-cell microscopy measurements as well by embedding cells from blood cultures in agar to detect the growth of bacterial aggregates. In the fASTest system10, an assessment of antibiotic susceptibility can be performed in 30 minutes. However, the technique is based on growth and works only if there are a relatively large number (104) of cells. If the bacteria are slow-growing (common in clinical samples), the assessment can take much longer. In the near future, other single-cell microscopy based platforms should be expected to emerge, as these represent the natural evolution to higher sensitivity. The coupling of single-cell microscopy with a growth-independent method represents a significant evolution of antibiotic susceptibility assessment.

PCR techniques have emerged as method for antibiotic susceptibility testing over the past decade. The major advantage of this method is the high sensitivity of the method, thus requiring a low volume of sample. The major disadvantage of the method is that it is DNA-based, which does not provide a functional assessment of antibiotic susceptibility. In particular, the presence of a gene or gene fragment does not indicate that the sequence is encoded or properly expressed, which severely limits the interpretation of the data for the purpose of antibiotic susceptibility determination. The method is very effective at early identification of bacteria, and the device built by T2 Biosystems ($260-$331 million valuation) can identify the presence of sepsis-causing bacteria in 4 hours using a combination of PCR and nuclear magnetitic resonance. T2 has the distinction of being the only FDA-approved method to perform this assessment. While this method is effective at determining the presence of pathogens, it is emphasized that it does not provide a functional test for antibiotic susceptibility.

Smarticles Technology, which was recently acquired by Roche (11), is a notable divergent technology. This platform utilizes phage technology to deliver custom DNA templates that encode luciferase to bacterial cells. Resistant bacteria produce the light-producing enzyme luciferase whereas susceptible bacteria do not produce this. The drawbacks of this method include that bacteriophage infections are strain-specific and bacteria must have compatible phage receptors. As many bacterial pathogens are phage resistant (this is a significant issue in the phage therapy community), Smarticles Technology will have to overcome this limitation. In addition, it is unclear how light emitted from only a few cells will be detected, given that the detection of luciferase in the lab requires many cells (>104 CFU of bacteria).

We have developed a device based on the principles of epi-fluorescence and time-correlated FLIM containing a collection apparatus that correlates the excitation pulse and data collection with nanosecond resolution. The current version of the device sits on the benchtop and is approximately 2 ft.×3 ft.×3 ft (w×d×h). The device can be further compacted.

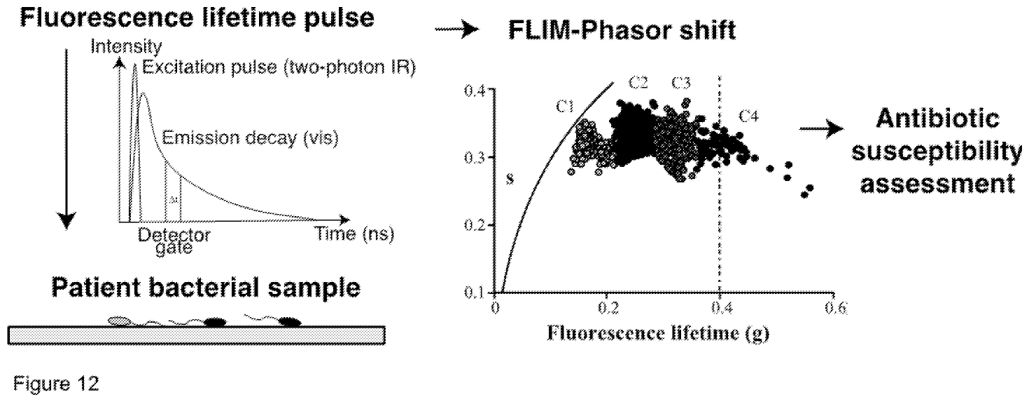

The process of antibiotic susceptibility testing involves: isolation of bacteria from patient samples, immobilization of bacteria on agarose pads that contain different antibiotics supplied above the accepted minimum inhibitory concentration from the major classes commonly used in clinical settings including amoxicillin (penicillin-type), cephalexin (cephalosporin), erythromycin (macrolide), ciprofloxacin (fluoroquinolone), trimethoprim (sulfonamide), tetracycline, and gentamicin (aminoglycoside), imaging using the FLIM-AST device immediately and at 15-minute intervals for 2 hours to assess metabolic kinetics in response to antibiotics (FIG. 12).

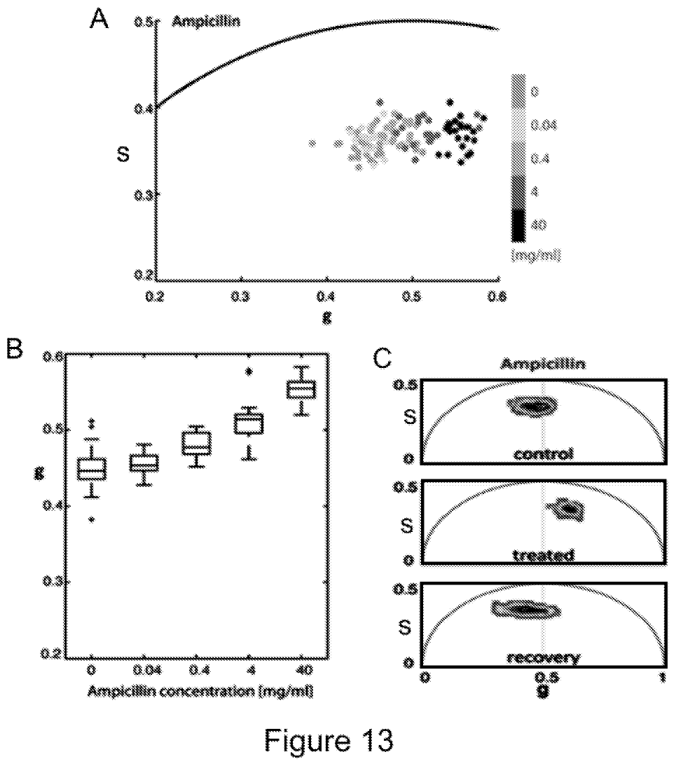

The antibiotic susceptibility determination is performed by comparing FLIM-phasor profiles during the 2-hour incubation using software. Cells that are susceptible to antibiotics will exhibit an increase in a shift towards higher g values, as observed in the preliminary data (FIG. 13)¹². Bacteria that are resistant to antibiotics will not exhibit a shift towards higher g values, similar to the recovered cells (FIG. 13)¹².

The FLIM microscope uses an 80 MHz ultrafast Ti:Sapphire Mai Tai laser (Spectra-physics, Santa Clara, Calif.) set at 740 nm for excitation. The setup used a 690-nm SP dichroic-460/80 nm filter pair for separating emission and a PlanApo N Olympus oil immersion 60× (1.42 NA) objective (Olympus, Waltham, Mass.), which is capable of bacterial single-cell resolution. An H7422P-40 photomultiplier tube module (Hamamatsu, Bridgewater, N.J.) and A320 FastFLIM Box (ISS, Champaign, Ill.) were used to measure fluorescence lifetime. Image acquisition was controlled by SimFCS software (Laboratory for Fluorescence Dynamics, Irvine, Calif.).

Fluorescence lifetime imaging was performed at room temperature. The microscope was calibrated before each session by setting the fluorescence lifetime obtained for 10 μM rhodamine 110 (Sigma, St. Louis, Mo.) to 4.0 ns. The laser power was set to 20%. Sub-cellular fluorescence lifetimes (FIG. 1A) were collected using the 20× optical zoom mode at a rate of 1.7 sec/frame. All other P. aeruginosa measurements were performed using the 6× optical zoom mode using the same frame rate. For all measurements, 40 sequential frames were acquired to generate a single FLIM image.

Masks for bacterial cells were created through SimFCS using fluorescence intensity images and image intensity thresholding. Fluorescence lifetimes within the masked areas were transformed using

$${g}_{i , j} ( ω ) = \text{?} {I}_{i , j} ( t ) cos ( ω t ) dt \text{/} \text{?} {I}_{i , j} ( t ) dt and {s}_{i , j} ( ω ) = \text{?} {I}_{i , j} ( t ) sin ( ω t ) dt \text{/} \text{?} {I}_{i , j} ( t ) dt , \\ \text{?} \text{indicates text missing or illegible when filed}$$

where I(t) is the fluorescence intensity decay, ω is the laser repetition angular frequency, and the indexes i and j identify a pixel of the image.

To measure the fluorescence lifetimes of cells grown using individual carbon sources (FIGS. 12 and 13B), individual colonies of P. aeruginosa were inoculated into culture tubes containing minimal medium A supplemented with 0.2% glucose, grown overnight to saturation, diluted 1:100 into modified minimal medium A containing sodium pyruvate, sodium citrate, glycerol, or glucose at a concentration of 0.2%, cultured to an OD₆₀₀ of 0.2, harvested, immobilized on a 1% agarose (Invitrogen, Carlsbad, Calif.) pad placed between a microscope slide (Fisher Scientific, Pittsburgh, Pa.) and a cover glass (VWR, Radnor, Pa.), as described previously (10), and imaged using fluorescence lifetime microscopy. For FIG. 2B, cells with fluorescence intensities below a value of 1.0, as reported by the SimFCS software, were excluded from the analysis. For experiments involving antimycin A, cultures were grown to saturation in modified minimal medium A containing 0.2% citrate, diluted 1:100 into the same medium, supplemented with 1% ethanol or 10 μM antimycin A (Sigma, St Louis, Mo.) dissolved in 1% ethanol, cultured to an OD₆₀₀ of 0.2, and immediately measured for fluorescence lifetime.

To measure the fluorescence lifetimes of planktonic and surface-attached P. aeruginosa populations (FIGS. 3E and 4A), individual colonies were inoculated into culture tubes containing PS:DB, cultured overnight to saturation, diluted 1:100 into 35 mm glass-bottom dishes (MatTek Corporation, Ashland, Mass.) containing the same medium, cultured between 1 to 8 hours, and harvested. Planktonic and surface-attached cells were isolated as described previously (2). Agar pads were made using Bacto agar at a concentration of 1% and DB buffer, and were cut into 1.5×1.5 cm squares. Planktonic cells were isolated from cultures grown in glass-bottom dishes, transferred onto a new dish, and immobilized by placing a pad on top of the cells (FIG. 11A). Surface-attached cells were isolated by aspirating the supernatant from the dish, washing the dish with DB buffer to remove planktonic cells, and placing an agar pad on the dish surface. Cells were imaged for fluorescence intensity and fluorescence lifetime using the FLIM device immediately after the pad was placed on top of the cells. Fluorescence lifetimes for pyocyanin and pyoverdine (FIG. 7C) were measured using solutions of 6.2 mM pyocyanin (P0046, Sigma, St Louis, Mo.) in DMSO and 5 mg/mL pyoverdine (P8124, Sigma, St. Louis, Mo.) in deionized water.

This device takes advantage of the single-cell microscopy, which has been proven in the QuantaMatrix, Accelerate Phenosystem, and fASTest systems and represents the next step in AST technology. This FLIM-AST device provides a quantum leap forward in AST technology by coupling single-cell detection with a non-destructive measurement of metabolic activity through FLIM. This measurement provides a much faster detection of antibiotic susceptibility than growth alone and the assessment does not require overcoming any complex biological processing of samples, which are significant barriers in the Accelerate Phenosystem and Smarticles systems.

### REFERENCES

1. Gould, I. M. & Bal, A. M. Virulence 4, 185-191 (2013).
2. Golkar, Z., et al. The Journal of Infection in Developing Countries 8, (2014).
3. Ventola, C. L. P T 40, 277-283 (2015).
4. Sengupta, S., et al. Frontiers in Microbiology 4, (2013).
5. Rossolini, G. M., et al. Current Opinion in Pharmacology 18, 56-60 (2014).
6. Tacconelli, E. et al. The Lancet Infectious Diseases 18, 318-327 (2018).
7. Puttaswamy, S., et al. Archives of Clinical Microbiology 09, (2018).
8. Wang, H. Y., et al. Clinical Microbiology and Infection 23, 333.e1-333.e7 (2017).
9. Choi, J. et al. Scientific Reports 7, (2017).
10. Baltekin, Ö., et al. Proceedings of the National Academy of Sciences 114, 9170-9175(2017).
11. Roche gobbles Smarticles. Nature Biotechnology 33, 1012-1012 (2015).
12. Bhattacharjee, A., et al. Scientific Reports 7, (2017).

### Example 4: FLIM-AST (or FAST) Extended to Additional Bacteria and Antibiotics

This Example shows further performance of the FLIM-AST with laboratory strains of Gram-positive and Gram-negative bacteria. We performed FLIM measurements using our FLIM AST (FAST) device of antibiotic-sensitive and antibiotic-resistant E. coli lab strains MG1655 and MC4100, P. aeruginosa strain PA14, and S. aureus strain RN4220 using a broad range of commonly-used antibiotics including tetracycline, ampicillin, carbenicillin, chloramphenicol, spectinomycin, gentamycin, and kanamycin and using a range of concentrations for each drug. For each antibiotic and strain combination, the minimum inhibitory concentration for each antibiotic was determined through measurements of optical densities of cultures. We treated bacterial strains using a range of concentrations starting from below-MIC to above-MIC concentrations for 1 hour.

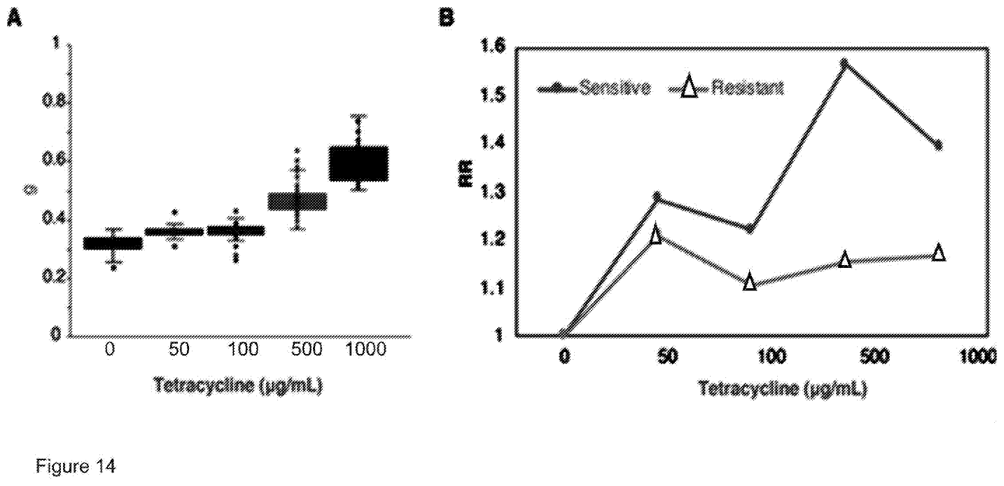

We computed g values using the FLIM phasor method. Consistent with our predictions, we observed a significant change in g value following treatment by many antibiotics. In particular, tetracycline exhibited the greatest changes in g values after 1 hour of treatment (FIG. 14A).

To determine a quantitative assessment of resistance, we computed a metric for the degree of antibiotic susceptibility for a given bacterial strain. Our experiments determined that the residual ratio (RR) number was a good indicator of antibiotic resistance:

$$RR = \frac{{( {g}_{t 1} - {g}_{t 0} )}_{treated}}{{( {g}_{t 1} - {g}_{t 0} )}_{untreated}} ,$$

where the g value is obtained using the phasor method to fluorescence lifetime imaging microscopy (FLIM) and t0 and t1 are the measurement times at the beginning of the measurement or at the end of the experiment, respectively. This metric provides an assessment of the shift in metabolic activity over the course of the experiment.

Our experiments determined that a distinction between antibiotic resistant and susceptible bacteria could be assessed for a range of antibiotics after 1 hour. The most clear assessment was made using tetracycline with E. coli (FIG. 14B). Here the change in metabolic activity is reflected as an increase in RR value beginning at 50 μg/mL of the drug and becoming even more distinct as the concentration of drug increased (FIG. 14B).

While the most clear assessment could be made for tetracycline, the assessment was less clear for other antibiotics. An unanticipated challenge was that antibiotic-resistant strains were never truly resistant to an antibiotic. At sufficiently high concentrations of antibiotics, even resistant strains were susceptible to antibiotics. Thus, the determination of antibiotic susceptibility became a relative determination—some strains were more resistant than others, but none were resistant at the highest antibiotic concentrations. The output of the FAST device could therefore be focused on the determination of the concentration of antibiotic at which a change in metabolic activity is observed, rather than if a strain is simply resistant or susceptible. The use of a larger number of antibiotic concentrations can be used to increase the resolution of how susceptible a bacterial strain is to an antibiotic. Our measurements were performed after 1 hour of incubation with antibiotic. Our data using a complementary metabolic assay (through measurement of respiration) suggested that metabolic changes occur much earlier than 1 hour. Thus, one can begin to assess antibiotic susceptibility at earlier times, such as 15 or 30 minutes. We note that the movement to an earlier measurement will provide earlier assessment of antibiotic susceptibility, which is the major unmet challenge in the field.

We performed antibiotic susceptibility assessments using 3 clinical E. coli strains from septic patients and 10 clinical S. aureus clinical strains. The technique again determined the resistance to tetracycline in E. coli strains. The assessment for tetracycline susceptibility was inconclusive in S. aureus and using other antibiotics in S. aureus and E. coli. We believe that the assessment time may need to be performed at earlier time points.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for inhibiting bacterial virulence, the method comprising exposing a site containing or suspected of containing virulent bacteria to a carbon source, wherein the carbon source produces a low g value.

2. A method for inhibiting bacterial virulence in a subject in need thereof, the method comprising administering a carbon source to the subject, wherein the carbon source produces a low g value.

3. The method of claim 1, wherein the bacteria comprise Pseudomonas aeruginosa.

4. The method of claim 2, wherein the carbon source is administered to the subject by topical application, injection into a wound site, or intravenous administration.

5. The method of claim 2, wherein the subject is a hospital or surgical patient.

6. The method of claim 2, wherein the subject is intubated, catheterized, or on a respirator.

7. The method of claim 2, wherein the subject is immunocompromised.

8. The method of claim 1, wherein the carbon source is pyruvate or citrate.

9. A device for testing antibiotic susceptibility of bacteria, the device comprising:
(a) a receiving surface adapted to receive and immobilize bacteria in contact with a test antibiotic;
(b) a fluorescence lifetime imaging microscopy (FLIM) apparatus that emits an excitation pulse of light directed at the receiving surface;
(c) a detector that collects time-correlated fluorescence emitted from individual bacteria immobilized on the receiving surface;
(d) an analyzer that correlates time-correlated fluorescence emitted from individual bacteria collected by the detector with the excitation pulse emitted by the FLIM apparatus to generate a FLIM-phasor profile; and
(e) an analyzer that correlates the FLIM-phasor profile generated in step (d) to the status of the antibiotic susceptibility of the bacteria.

10. The device of claim 9, wherein the detector collects fluorescence with nanosecond resolution.

11. A method of testing antibiotic susceptibility of bacteria isolated from a patient sample, the method comprising:
(a) immobilizing bacteria isolated from a patient sample onto a receiving surface;
(b) measuring the FLIM signatures at an initial time point upon contacting the immobilized bacteria with a test antibiotic and at a plurality of intervals for 30 minutes to 1 hour;
(c) directing a series of nanosecond excitation pulses of light at the immobilized bacteria;
(d) collecting time-correlated fluorescence emitted from individual bacteria immobilized on the receiving surface;
(e) generating FLIM-phasor profiles by taking the sine and cosine transform of the fluorescence intensity decays, thereby generating s and g values; and
(f) comparing FLIM-phasor profiles obtained before and after the contacting of step (b); wherein a change in the g value upon contact with a test antibiotic indicates bacterial susceptibility to the test antibiotic.

12. The method of claim 11, wherein the test antibiotic is selected from the group consisting of: amoxicillin (penicillin-type), cephalexin (cephalosporin), erythromycin (macrolide), ciprofloxacin (fluoroquinolone), trimethoprim (sulfonamide), tetracycline, and gentamicin (aminoglycoside).

13. The method of claim 11, wherein steps (c)-(e) of the method are repeated at intervals of 10-20 minutes for 1-3 hours after contacting the bacteria with test antibiotic.

14. The method of claim 13, wherein steps (c)-(e) of the method are repeated at intervals of 15 minutes for 2 hours after contacting the bacteria with test antibiotic.

15. A system for testing antibiotic susceptibility of bacteria, the system comprising a user device comprising a hardware processor that is programmed to generate and analyze FLIM-phasor profiles as recited in claim 11.

16. A non-transitory computer-readable medium containing computer executable instructions that, when executed by a processor, cause the processor to generate and analyze FLIM-phasor profiles as recited in claim 11.

17. The method of claim 2, wherein the bacteria comprise Pseudomonas aeruginosa.

18. The method of claim 2, wherein the carbon source is pyruvate or citrate.
